(19) United States
(12) Patent Application Publication
(10) Pub. No.: US 2010/0190162 A1
(43) Pub. Date: Jul. 29, 2010

(54) METHODS OF USING SINGLE NUCLEOTIDE POLYMORPHISMS IN THE TL1A GENE TO PREDICT OR DIAGNOSE INFLAMMATORY BOWEL DISEASE

(75) Inventors: Jerome I. Rotter, Los Angeles, CA (US); Kent D. Taylor, Ventura, CA (US); Marla Dubinsky, Los Angeles, CA (US); Stephan R. Targan, Santa Monica, CA (US)

Correspondence Address: DAVIS WRIGHT TREMAINE LLP/Los Angeles, 865 FIGUEROA STREET, SUITE 2400, LOS ANGELES, CA, 90017-2566, US

(73) Assignee: CEDARS-SINAI MEDICAL CENTER, Los Angeles, CA (US)

(21) Appl. No.: 12/528,055

(22) PCT Filed: Feb. 26, 2008
(86) PCT No.: PCT/US08/55020

§ 371 Date: Mar. 5, 2010

Related U.S. Application Data
(60) Provisional application No. 60/891,578, filed on Feb. 26, 2007.

(51) Int. Cl. C12Q 1/68

(52) U.S. Cl. 435/6

(57) ABSTRACT

This invention provides methods of diagnosing or predicting susceptibility to Inflammatory Bowel Disease by determining the presence or absence of genetic variants in the TL1A gene. In one embodiment, a method of the invention is practiced by determining the presence or absence of TL1A production following Fc-gamma-R activation. In another embodiment, the invention provides methods of treatment of inflammatory bowel disease by inhibition of TL1A.

### FIELD OF THE INVENTION

The invention relates generally to the fields of inflammation and autoimmunity and autoimmune disease and, more specifically, to genetic methods for diagnosing inflammatory bowel disease, Crohn's disease, and other autoimmune diseases.

### BACKGROUND

All publications herein are incorporated by reference to the same extent as if each individual publication or patent application was specifically and individually indicated to be incorporated by reference. The following description includes information that may be useful in understanding the present invention. It is not an admission that any of the information provided herein is prior art or relevant to the presently claimed invention, or that any publication specifically or implicitly referenced is prior art.

Crohn's disease (CD) and ulcerative colitis (UC), the two common forms of idiopathic inflammatory bowel disease (IBD), are chronic, relapsing inflammatory disorders of the gastrointestinal tract. Each has a peak age of onset in the second to fourth decades of life and prevalences in European ancestry populations that average approximately 100-150 per 100,000 (D. K. Podolsky, N Engl J Med 347, 417 (2002); E. V. Loftus, Jr., Gastroenterology 126, 1504 (2004)). Although the precise etiology of IBD remains to be elucidated, a widely accepted hypothesis is that ubiquitous, commensal intestinal bacteria trigger an inappropriate, overactive, and ongoing mucosal immune response that mediates intestinal tissue damage in genetically susceptible individuals (D. K. Podolsky, N Engl J Med 347, 417 (2002)). Genetic factors play an important role in IBD pathogenesis, as evidenced by the increased rates of IBD in Ashkenazi Jews, familial aggregation of IBD, and increased concordance for IBD in monozygotic compared to dizygotic twin pairs (S. Vermeire, P. Rutgeerts, Genes Immun 6, 637 (2005)). Moreover, genetic analyses have linked IBD to specific genetic variants, especially CARD15 variants on chromosome 16q12 and the IBD5 haplotype (spanning the organic cation transporters, SLC22A4 and SLC22A5, and other genes) on chromosome 5q31 (S. Vermeire, P. Rutgeerts, Genes Immun 6, 637 (2005); J. P. Hugot et al., Nature 411, 599 (2001); Y. Ogura et al., Nature 411, 603 (2001); J. D. Rioux et al., Nat Genet 29, 223 (2001); V. D. Peltekova et al., Nat Genet 36, 471 (2004)). CD and UC are thought to be related disorders that share some genetic susceptibility loci but differ at others.

The replicated associations between CD and variants in CARD15 and the IBD5 haplotype do not fully explain the genetic risk for CD. Thus, there is need in the art to determine other genes, allelic variants and/or haplotypes that may assist in explaining the genetic risk, diagnosing, and/or predicting susceptibility for or protection against inflammatory bowel disease including but not limited to CD and/or UC.

### SUMMARY OF THE INVENTION

Various embodiments provide methods of diagnosing susceptibility to a subtype of Crohn's Disease in an individual, comprising determining the presence or absence of one or more risk variants at the TNFSF15 locus in the individual, where the presence of one or more risk variants at the TNFSF15 locus is diagnostic of susceptibility to the subtype of Crohn's Disease. In other embodiments, the individual is a child. In other embodiments, the subtype is associated with the absence of NOD2 risk variants, or further comprises complicated small bowel disease phenotype, or internal penetrating and/or stricturing disease phenotype. In other embodiments, one of the one or more risk haplotypes at the TNFSF15 locus in the individual is haplotype A, and comprises one or more variant alleles selected from SEQ. ID. NO.: 3, SEQ. ID. NO.: 4, SEQ. ID. NO.: 5, SEQ. ID. NO.: 6, and SEQ. ID. NO.: 7.

Other various embodiments provide methods of determining in an individual a low probability relative to a healthy individual of developing inflammatory bowel disease, comprising determining the presence or absence of one or more protective haplotypes at the TNFSF15 locus, where the presence of one or more protective haplotypes at the TNFSF15 locus is diagnostic of the low probability relative to the healthy individual of developing inflammatory bowel disease. In other embodiments, the individual is a child, and/or non-Jewish. In other embodiments, the inflammatory bowel disease further comprises complicated small bowel disease phenotype, internal penetrating and/or stricturing disease phenotype, Crohn's Disease, and/or ulcerative colitis. In other embodiments, one of the one or more protective haplotypes at the TNFSF15 locus is haplotype B, and/or comprise one or more variant alleles selected from SEQ. ID. NO.: 3, SEQ. ID. NO.: 4, SEQ. ID. NO.: 5, SEQ. ID. NO.: 6, and SEQ. ID. NO.: 7.

In other embodiments, there are methods of treating inflammatory bowel disease in a mammal, comprising administering a therapeutically effective amount of TL1A antagonist. The TL1A antagonist can also comprise a TL1A antibody. In other embodiments, the mammal is a mouse or human. In another embodiment, the TL1A antagonist comprises SEQ. ID. NO. 2. The inflammatory bowel disease may also further comprise Crohn's Disease.

Various embodiments provide methods of treating inflammation in a mammal, comprising determining the presence of one or more risk variants at the TL1A locus in the mammal, and administering a therapeutically effective amount of a TL1A antagonist. The inflammation may also be associated with a chronic inflammatory disease, which in turn may include rheumatoid arthritis, multiple sclerosis, and/or psoriasis.

Other embodiments provide methods of treating chronic colitis in a mammal, comprising administering a therapeutically effective amount of TL1A antagonist, which may be anti-TL1A mAb. In other embodiments, the mammal is a mouse, or a human.

Other embodiments provide methods of diagnosing susceptibility to a subtype of inflammatory bowel disease in a mammal, comprising determining the presence or absence of TL1A expression, where the presence of TL1A expression is diagnostic of susceptibility to the subtype of inflammatory bowel disease in the mammal. The mammal may be a human, or a mouse. In other embodiments, the TL1A expression is a result of Fc-gamma R stimulation

Other embodiments provide methods of treating a condition in a mammal associated with up-regulation of T_{H}1 and/or T_{H}17 activation, comprising administering a therapeutically effective amount of inhibitor of TL1A expression. The inhibitor of TL1A expression may comprise anti-TL1A mAb.

Other features and advantages of the invention will become apparent from the following detailed description, taken in conjunction with the accompanying drawings, which illustrate, by way of example, various embodiments of the invention.

### BRIEF DESCRIPTION OF THE FIGURES

Exemplary embodiments are illustrated in referenced figures. It is intended that the embodiments and figures disclosed herein are to be considered illustrative rather than restrictive.

FIG. 1 depicts the genotype (Taqman) for 3 NOD2 variants (SNP 8, 12, 13) and 5 polymorphisms of TNFSF15 (rs3810936, rs6478109, rs7848647, rs7869487). The genotype was constructed from DNA collected from 253 pediatric CD patients enrolled from sites WRAPID.

FIG. 2 depicts haplotypes constructed using Phase 2.1.1. The main haplotype structure of TNFSF15 gene is depicted, with nucleotides described for the corresponding SNP position.

FIG. 3 depicts a chart describing a lack of interaction between NOD2 and TNFSF15.

FIG. 4 depicts expression of TL1A mRNA by monocytes and mono-DC. Fresh monocytes from blood (left panel) and mono-DC (right panel) were stimulated by plate-bound IgG (), LPS (▪), Pam3CSK4 (♦), CBir1 Flagellin (⋄), and IFN-gamma (x). TL1A transcript was quantitated by real-time RT-PCR and expressed as percent actin transcript level (A and B). One representative experiment out of two time-course experiments is shown.

FIG. 5 depicts TL1A mRNA induction by stimulation of fresh monocytes (upper panel, n=5) and mono-DC (lower panel, n=4) by plate-bound IgG for 6 hours. TL1A transcript was quantitated by real-time RT-PCR and expressed as percent Beta-actin transcript level.

FIG. 6 depicts expression of soluble TL1A by monocytes and mono-DC. Fresh monocytes from blood (left panel) and mono-DC (right panel) were stimulated by plate-bound IgG (), LPS (▪), Pam3CSK4 (♦), CBir1 Flagellin (⋄), and IFN-gamma (x). TL1A in supernatants was quantified by ELISA (A and B). One representative experiment out of two time-course experiments is shown.

FIG. 7 depicts TNF-α and IL-6 secretion by monocytes and DCs upon stimulation with IFN-gamma, TLR ligands, and immune complexes. Monocytes or DCs were stimulated with IFN-gamma (10 ng/ml, LPS (50 ng/ml), Pam3CSK4 (300 ng/ml), Flagellin (10 μg/ml), and plate-bound IgG for 6 h (TNF-alpha) or 18 h (IL-6), respectively. TNF-α (C and D) and IL-6 (A and B) response was measured in supernatants by ELISA. Data shown are means±SD of one representative experiment out of two independent experiments.

FIG. 8 depicts induction of cell surface TL1A on monocytes (left panels) and mono-DC (right panels) without stimulation (A and B) and after stimulation with LPS(C and D), Pam3CSK4 (E and F), and plate-bound IgG (16 h) (G and H). Cells were stained for flow cytometry with a TL1A specific MAb (shaded) or isotype control (unshaded). Representative of 4 experiments with monocytes and 4 experiments with dendritic cells, respectively.

FIG. 9 depicts TL1A enhances IFN-gamma production by CD4+ T cells in co-cultures with monocytes. A: CD4+ T cells were isolated and incubated overnight with IL-12 and IL-18. The next day, T cells were either cultured alone or co-cultured with monocytes that had been pre-incubated with or without IC overnight. After 48 h supernatants were harvested and analyzed for IFN-gamma production by ELISA. B: Co-cultures of CD4+ T cells and IC treated monocytes were left untreated or incubated with anti-TL1A or control antibodies. After 48 h supernatants were harvested and analyzed for IFN-gamma production by ELISA. Representative of three experiments with similar results is shown.

FIG. 10 (A)-(C) depicts a chart of data generated under the noted cell types and conditions.

FIG. 11 (A)-(D) depicts a chart of data generated under the noted cell types and conditions.

FIG. 12 depicts a chart summarizing data results. The average of each haploid type is given. The graphs depict data generated and previously described.

FIG. 13 (A)-(E) depicts charts summarizing data generated.

FIG. 14 depicts t-test results and demonstrate statistical significance of data generated.

FIG. 15 depicts a graph demonstrating percentage of body weight over time with DSS models.

FIG. 16 depicts a graph of colon length for control compared to DSS, and a picture of sample.

FIG. 17 depicts graphs demonstrating cell numbers of MLN and LP cell, each comparing control vs. DSS.

FIG. 18 depicts graphs demonstrating amount of IFN-gamma and IL-17 production, each with MLN and LP cells.

FIG. 19 depicts a graph of FACS analysis in MLN.

FIG. 20 depicts a graph of FACS analysis in LP and Spleen cells.

FIG. 21 depicts a graph of RT-PCR results in colon tissue.

FIG. 22 depicts a graph of RT-PCR results in MLN.

FIG. 23 depicts a graph demonstrating percentage of body weight over time with DSS.

FIG. 24 depicts a graph of colon length for Hamster IgG vs. mTL1A antibody, and a picture of sample.

FIG. 25 depicts a graph of cell numbers for both MLN and LP cells. Each depict cell numbers for both Hamster IgG and mTL1A antibody.

FIG. 26 depicts graphs of IFN-gamma production of MLN and IFN-gamma production of LP cells.

FIG. 27 depicts graphs of IL-17 production of MLN and IL-17 production of LP cells.

FIG. 28 depicts graphs of IFN-gamma in MLN of DSS mice, and IL-17 in MLN of DSS mice. Each demonstrate results with no stimulation, TL1A concentration of 10 ng/ml, and TL1A concentration of 50 ng/ml.

FIG. 29 depicts graphs of IFN-gamma production of MLN and IFN-gamma production in MLN of DSS mice.

FIG. 30 depicts graphs of IL-17 of MLN, and IL-17 of MLN of DSS mice.

FIG. 31 depicts graphs of IFN-gamma in LP cells of DSS mice, and IL-17 in LP cells of DSS mice.

FIG. 32 depicts graphs of CD3/CD28 stimulation. The graphs describe IFN-gamma production in MLN and IFN-gamma in MLN of DSS mice.

FIG. 33 depicts graphs of CD3/CD28 stimulation. The graphs describe IFN-gamma in LP cells and IFN-gamma in LP of DSS mice.

FIG. 34 depicts graphs of CD3/CD28 stimulation. The graphs describe IL-17 in MLN, and IL-17 in MLN of DSS mice.

FIG. 35 depicts graphs of CD3/CD28 stimulation. The graphs describe IL-17 production in LP cells, and IL-17 in LP of DSS mice.

FIG. 36 depicts graphs of IFN-gamma in MLN of DSS mice, and IL-17 in MLN of DSS mice.

FIG. 37 depicts a chart demonstrating neutralizing TL1A antibodies attenuates TH1 and TH17 responses in vitro. Mononuclear cells from MLN of DSS-treated mice were cultured in the presence of IL-12 plus TL1A or IL-23 plus TL1A with increasing concentrations of neutralizing TL1A mAb (0, 2, 5, or 10 mg/mL) for 72 h. The left panel depicts IFN-gamma production of MLN stimulated with IL-12 and TL1A in the presence of increasing concentrations of neutralizing TL1A mAb (n=4 per group). The right panel depicts IL-17 production of MLN stimulated with IL-23 and TL1A in the presence of increasing concentrations of neutralizing TL1A mAb (n=4 per group).

FIG. 38 depicts a chart demonstrating that the number of infiltrating cells is significantly reduced in mice treated with anti-TL1A mAb. Mononuclear cells numbers from MLN in the left panel and LPMC in the right panel of DSS treated mice receiving either anti-TL1A or control Ab (n=10 per group).

FIG. 39 depicts a chart demonstrating that treatment with anti-TL1A mAb significantly improves established chronic colitis. Mononuclear cells numbers from MLN in the left panel and LPMC of the right panel of DSS treated mice receiving either anti-TL1A or control Ab (n=5 per group). Treatment with antibodies was started after 2 cycles of DSS.

FIG. 40 depicts an example of staining demonstrating that treatment with anti-TL1A mAb significantly improves established chronic colitis. H and E staining of representative colons from DSS-treated mice receiving anti-TL1A mAb treatment on the right panel, with original magnification ×200, or control IgG of the left panel, with original magnification ×100 treatment.

FIG. 41 depicts a chart demonstrating that neutralizing IL-23R antibodies attenuates Th17 responses in vitro. Mononuclear cells from MLN of DSS-treated mice were cultured in the presence of IL-23 plus TL1A with increasing concentrations of neutralizing IL-23 and TL1A in the presence of increasing concentrations of neutralizing TL1A mAb was measured by ELISA (n=4 per group).

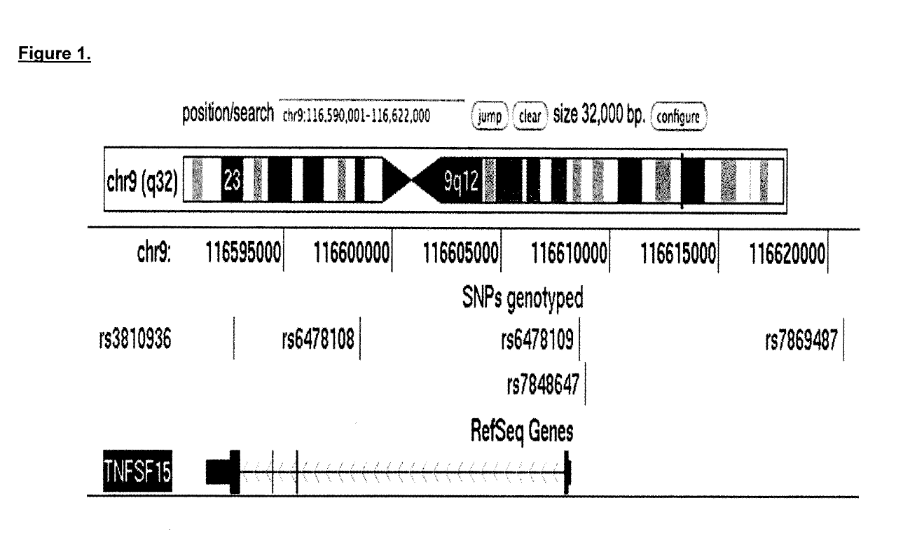

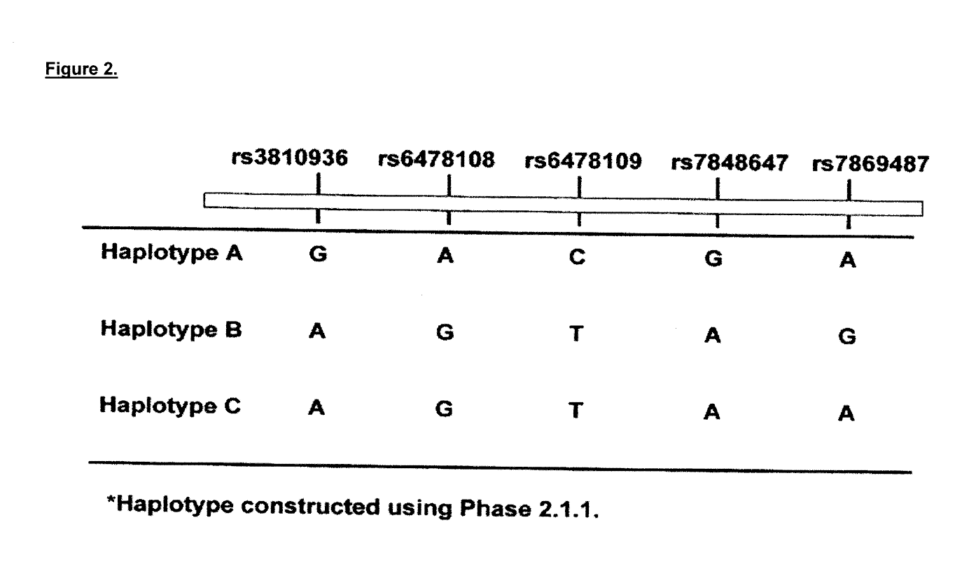

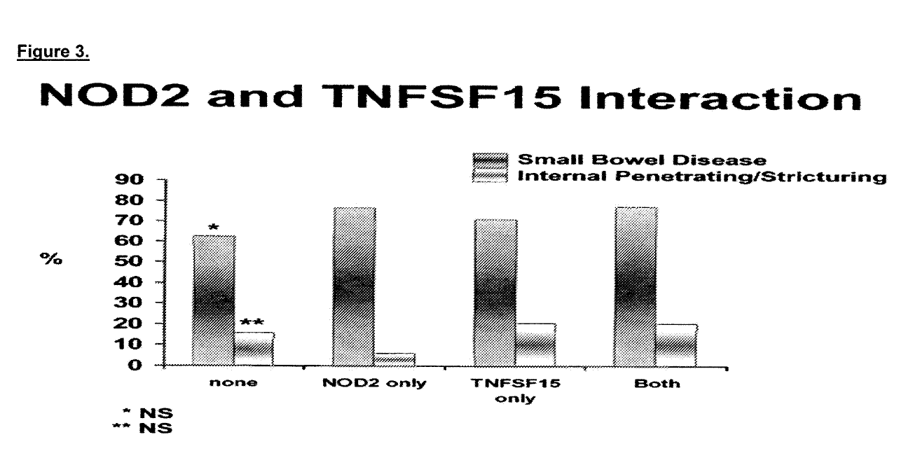

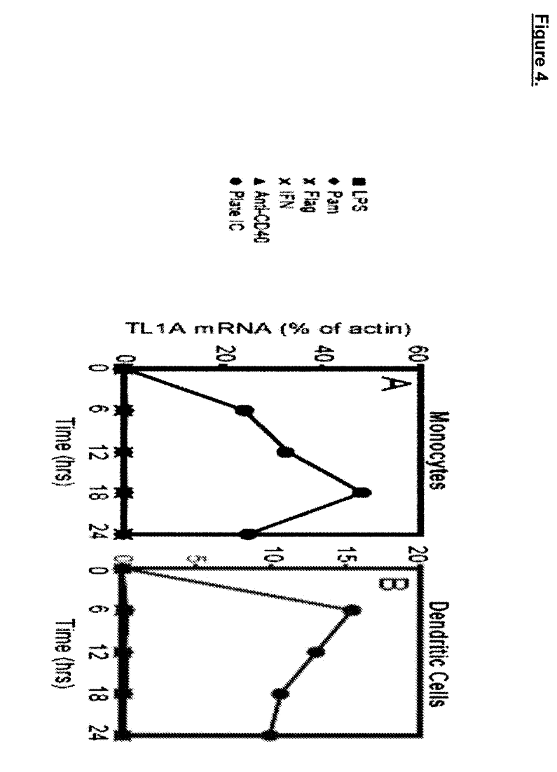

### DESCRIPTION OF THE INVENTION

All references cited herein are incorporated by reference in their entirety as though fully set forth. Unless defined otherwise, technical and scientific terms used herein have the same meaning as commonly understood by one of ordinary skill in the art to which this invention belongs. Singleton et al., Dictionary of Microbiology and Molecular Biology 3^{rd} ed., J. Wiley & Sons (New York, N.Y. 2001); March, Advanced Organic Chemistry Reactions, Mechanisms and Structure 5^{th} ed., J. Wiley & Sons (New York, N.Y. 2001); and Sambrook and Russel, Molecular Cloning: A Laboratory Manual 3rd ed., Cold Spring Harbor Laboratory Press (Cold Spring Harbor, N.Y. 2001), provide one skilled in the art with a general guide to many of the terms used in the present application.

One skilled in the art will recognize many methods and materials similar or equivalent to those described herein, which could be used in the practice of the present invention. Indeed, the present invention is in no way limited to the methods and materials described.

“IBD” as used herein refers to Inflammatory Bowel Disease.

“CD” as used herein refers to Crohn's Disease.

“UC” as used herein refers to ulcerative colitis.

“Haplotype” as used herein refers to a set of single nucleotide polymorphisms (SNPs) on a gene or chromatid that are statistically associated.

“Protective variant” as used herein refers to an allele whose presence is associated with a decrease in susceptibility to an inflammatory disease, including but not limited to CD and UC, relative to an individual diagnosed with the disease.

“Risk variant” as used herein refers to an allele whose presence is associated with an increase in susceptibility to an inflammatory disease, including but not limited to CD and UC, relative to a healthy individual.

“Protective haplotype” as used herein refers to a haplotype sequence whose presence is associated with a decrease in susceptibility to an inflammatory disease, including but not limited to CD and UC, relative to an individual diagnosed with the disease.

“Risk haplotype” as used herein refers to a haplotype sequence whose presence is associated with an increase in susceptibility to an inflammatory disease, including but not limited to CD and UC, relative to a healthy individual.

As used herein, the term “sero-reactivity” means positive expression of an antibody.

As used herein, the term “biological sample” means any biological material from which nucleic acid molecules can be prepared. As non-limiting examples, the term material encompasses whole blood, plasma, saliva, cheek swab, or other bodily fluid or tissue that contains nucleic acid.

“TNFSF15” as used herein also means “TL1A,” “TNF super family member 15,” and “Tumour necrosis factor super family 15.”

As used herein, “haplotype 16” also can be described as “haplotype A,” or “risk haplotype.”

As used herein, “haplotype 1” also can be described as “haplotype B,” and “haplotype 1-1,” and “haplotype non-16,” and “protective haplotype.”

As used herein, R702W, G908R, and 1007fs variant alleles are also described as SNP 8, 12, and 13, respectively, as well as R675W, G881R, and 3020insC, respectively.

As used herein, “haplotype A” means that nucleotides G, A, C, G, and A are the variant alleles of the TNFSF15 SNPs rs3810936, rs6478108, rs6478109, rs7848647, rs7869487, respectively. In other words, in haplotype A, the variant allele of rs3810936 is G, the variant allele of rs6478108 is A, the variant allele of rs6478109 is C, the variant allele of rs7848647 is G, and the variant allele of rs7869487 is A.

As used herein, “haplotype B” means that nucleotides A, G, T, A, and G are the variant alleles of the TNFSF15 SNPs rs3810936, rs6478108, rs6478109, rs7848647, rs7869487, respectively. In other words, in haplotype B, the variant allele of rs3810936 is A, the variant allele of rs6478108 is G, the variant allele of rs6478109 is T, the variant allele of rs7848647 is A, and the variant allele of rs7869487 is G.

As disclosed herein, an example of a TNFSF15 genetic sequence is described as SEQ. ID. NO.: 1. An example of a TNFSF15 peptide sequence is described herein as SEQ. ID. NO.: 2.

Examples of the TNFSF15 polymorphisms rs3810936, rs6478108, rs6478109, rs7848647, rs7869487 are also described herein as SEQ. ID. NO.: 3, SEQ. ID. NO.: 4, SEQ. ID. NO.: 5, SEQ. ID. NO.: 6, and SEQ. ID. NO.: 7, respectively.

An example of a NOD2 genetic sequence is described as SEQ. ID. NO. 8. Examples of the NOD2 variants SNPs 8, 12 and 13, are also described herein as SEQ. ID. NO.: 9, 10 and 11, respectively.

The inventors performed an association study testing autosomal single nucleotide polymorphisms (SNPs) on the Illumina HumanHap300 Genotyping BeadChip. Based on these studies, the inventors have identified TNFSF15 as an inflammatory bowel disease gene. Additionally, the inventors found single nucleotide polymorphisms (SNPs) and haplotypes that are associated with increased or decreased risk for inflammatory bowel disease, including but not limited to CD and UC. These SNPs and haplotypes are suitable for genetic testing to identify at risk individuals and those with increased risk for complications associated with serum expression of Anti-Saccharomyces cerevisiae antibody, and antibodies to 12, OmpC, and Cbir. The detection of protective and risk SNPs and/or haplotypes in TNFSF15 may be used to identify at risk individuals, predict disease course and suggest the right therapy for individual patients. Additionally, the inventors have found both protective and risk allelic variants for Crohn's Disease and Ulcerative Colitis.

Based on these findings, embodiments of the present invention provide for methods of diagnosing and/or predicting susceptibility for or protection against inflammatory bowel disease including but not limited to Crohn's Disease and/or ulcerative colitis. Other embodiments provide for methods of prognosing inflammatory bowel disease including but not limited to Crohn's Disease and/or ulcerative colitis. Other embodiments provide for methods of treating inflammatory bowel disease including but not limited to Crohn's Disease and/or ulcerative colitis.

The methods may include the steps of obtaining a biological sample containing nucleic acid from the individual and determining the presence or absence of a SNP and/or a haplotype in the biological sample. The methods may further include correlating the presence or absence of the SNP and/or the haplotype to a genetic risk, a susceptibility for inflammatory bowel disease including but not limited to Crohn's Disease and ulcerative colitis, as described herein. The methods may also further include recording whether a genetic risk, susceptibility for inflammatory bowel disease including but not limited to Crohn's Disease and ulcerative colitis exists in the individual. The methods may also further include a prognosis of inflammatory bowel disease based upon the presence or absence of the SNP and/or haplotype. The methods may also further include a treatment of inflammatory bowel disease based upon the presence or absence of the SNP and/or haplotype.

In one embodiment, a method of the invention is practiced with whole blood, which can be obtained readily by non-invasive means and used to prepare genomic DNA, for example, for enzymatic amplification or automated sequencing. In another embodiment, a method of the invention is practiced with tissue obtained from an individual such as tissue obtained during surgery or biopsy procedures.

Novel Association Between TNFSF15 and Disease Phenotype

As disclosed herein, the inventors examined the association of the tumour necrosis factor super family 15 (TNFSF15) gene with disease phenotype and its interaction with NOD2 in pediatric CD patients.

As further disclosed herein, DNA was collected from 250 well characterized pediatric CD cases. Genotyping (TaqmanMGB) was performed for 3 CD-associated variants of NOD2, single nucleotide polymorphisms (SNPs) 8, 12, and 13 and for 5 polymorphisms of TNFSF15 gene, rs3810936, rs6478108, rs6478109, rs7848647, rs7869487. Haplotypes were constructed using PHASE 2.1.1. Phenotypes were determined by chart review blinded to genotype. Associations between candidate genes and disease phenotype (location and complicated behavior [internal penetrating and/or stricturing disease (IP/S)]) were determined.

As further disclosed herein, NOD2 variants were found in 32% of children and the carrier frequency of common alleles for TNFSF15 ranged from 90%-93%. The frequencies of the 2 most common TNFSF15 haplotypes, A and B, are 66% and 21%, respectively. NOD2 is not associated with disease location or behavior. In contrast, haplotype A of TNFSF15 is associated with small bowel (SB) (p=0.02) and IP/S (p=0.05). Carriage of the common allele of rs6478109 (−358) has the strongest association with both SB (p<0.001) and IP/S (p<0.02). There is no interaction between NOD2 and TNFSF15.

As further disclosed herein, an association is demonstrated between TNFSF15 and disease phenotype in CD patients. Carriage of the common haplotype is associated with complicated small bowel disease. Moreover, given its location, SNP rs6478109 plays a role in gene expression and this in turn modifies disease phenotype.

In one embodiment, the present invention provides methods of diagnosing and/or predicting susceptibility to Crohn's Disease in an individual by determining the presence or absence in the individual of haplotype A in the TNFSF15 gene. In another embodiment, the present invention provides methods of prognosis of Crohn's Disease in an individual by determining the presence or absence in the individual of haplotype A in the TNFSF15 gene. In another embodiment, the present invention provides methods of treatment of Crohn's Disease in an individual by determining the presence or absence in the individual of haplotype A in the TNFSF15 gene. In another embodiment, the absence of haplotype A is associated with an absence of NOD2 variants.

In one embodiment, the present invention provides methods of diagnosing and/or predicting protection against Crohn's Disease in an individual by determining the presence or absence in the individual of haplotype B in the TNFSF15 gene. In another embodiment, the present invention provides methods of prognosis of Crohn's Disease in an individual by determining the presence or absence in the individual of haplotype B in the TNFSF15 gene. In another embodiment, the present invention provides methods of treatment of Crohn's Disease in an individual by determining the presence or absence in the individual of haplotype B in the TNFSF15 gene.

In one embodiment, the present invention provides methods of diagnosing and/or predicting susceptibility to complicated small bowel disease in an individual by determining the presence or absence in the individual of haplotype A in the TNFSF15 gene. In another embodiment, the present invention provides methods of prognosis of complicated small bowel disease in an individual by determining the presence or absence in the individual of haplotype A in the TNFSF15 gene. In another embodiment, the present invention provides methods of treatment of complicated small bowel disease in an individual by determining the presence or absence in the individual of haplotype A in the TNFSF15 gene. In another embodiment, the absence of haplotype A is associated with an absence of NOD2 variants.

In one embodiment, the present invention provides methods of diagnosing and/or predicting protection against complicated small bowel disease in an individual by determining the presence or absence in the individual of haplotype B in the TNFSF15 gene. In another embodiment, the present invention provides methods of prognosis of complicated small bowel disease in an individual by determining the presence or absence in the individual of haplotype B in the TNFSF15 gene. In another embodiment, the present invention provides methods of treatment of complicated small bowel disease in an individual by determining the presence or absence in the individual of haplotype B in the TNFSF15 gene.

### TNFSF15 is an Ethnic Specific Gene

As disclosed herein, five SNPs that comprise two common haplotypes were genotyped in 572 Caucasian patients with Crohn's disease (CD), 377 Caucasian patients with Ulcerative Colitis (UC) and 216 ethnically-matched healthy controls, including both Jews and non-Jews.

As further disclosed herein, the risk haplotype (haplotype A) was not associated with CD or UC (88.3% in CD cases vs. 88.9% in controls, p=0.81; 88.9% in UC cases vs. 88.9% in controls, p=0.99). However, it was observed that there was an increased frequency of the protective haplotype (haplotype B) in Non-Jewish controls for both CD and UC (39.3% CD cases vs. 49.1% controls, p=0.03; 37.6% UC cases vs. 49.1% controls, p=0.02) with no such effect observed in the Jewish samples. There was an interactive effect between ethnicity and the protective haplotype in CD (p=0.04).

As further disclosed herein, a protective haplotype was observed, consisting of the minor alleles for all five markers, to have a higher frequency in the non-Jewish controls than in CD and UC. Additionally, the haplotype frequency was in the opposite direction, in the Jewish case-control panels (both CD and UC), leading the inventors to conclude (1) TNFSF15 is an IBD susceptibility gene, and (2) the disease susceptibility is ethnic specific.

In one embodiment, the present invention provides methods of diagnosing and/or predicting susceptibility to Crohn's Disease in an individual by determining the presence or absence of haplotype A in the TNFSF15 gene in a non-Jewish individual. In another embodiment, the present invention provides methods of prognosis of Crohn's Disease in an individual by determining the presence or absence of haplotype A in the TNFSF15 gene in a non-Jewish individual. In another embodiment, the present invention provides methods of treatment of Crohn's Disease by determining the presence or absence in the individual of haplotype A in the TNFSF15 gene in a non-Jewish individual.

In one embodiment, the present invention provides methods of diagnosing and/or predicting protection against Crohn's Disease by determining the presence or absence of haplotype B in the TNFSF15 gene in a non-Jewish individual. In another embodiment, the present invention provides methods of prognosis of Crohn's Disease by determining the presence or absence of haplotype B in the TNFSF15 gene in a non-Jewish individual. In another embodiment, the present invention provides methods of treatment of Crohn's Disease by determining the presence or absence of haplotype B in the TNFSF15 gene in a non-Jewish individual.

### The T Cell Co-Stimulator TL1A is Induced by Fcgamma Receptor Signaling in Human Monocytes and Dendritic Cells

As disclosed herein, TL1A/DR3 ligand/receptor pair mediates strong co-stimulation of Th1 cells. Activation of T and NK cells induces DR3 expression, permitting soluble recombinant TL1A to increase IFN-gamma production and proliferation of these cells. Gut T cells and macrophages express TL1A, especially in Crohn's disease (CD), and there is a strong association between CD and TL1A SNPs. Murine studies implicate TL1A in gut inflammation.

As further disclosed herein, to determine whether professional T cell activating cells can express TL1A, fresh blood monocytes and monocyte-derived DC were stimulated with various activating ligands, including TLR agonists, IFN-gamma, and immune complexes. Fc-gamma-R stimulation strongly induced TL1A mRNA in both cell types, which correlated with the detection of TL1A on the cell surface and in cell culture media. TLR agonists capable of inducing IL-6 and TNF-α in monocytes and DC did not induce surface nor soluble TL1A. Additionally, the inventors demonstrated that TL1A production in monocytes leads to enhancement of T cell responses. The induction of TL1A on APC via specific pathway stimulation shows a role for TL1A in Th1 responses to pathogens, and in CD.

In one embodiment, the present invention provides methods of diagnosing and/or predicting susceptibility to Crohn's Disease in an individual by determining the presence or absence of TL1A production following Fc-gamma-R activation. In another embodiment, the present invention provides methods of prognosis of Crohn's Disease in an individual by determining the presence or absence of TL1A production following Fc-gamma-R activation. In another embodiment, the present invention provides methods of treatment of Crohn's Disease by determining the presence or absence in the individual of TL1A production following Fc-gamma-R activation.

In one embodiment, the present invention provides methods of treatment of inflammatory bowel disease by inhibition of TL1A and/or Fc-gamma-R. In another embodiment, the present invention provides methods of treatment of inflammatory bowel disease by using antibodies to inhibit TL1A and/or Fc-gamma-R. In another embodiment, the present invention provides methods of treatment of inflammatory bowel disease by inhibition of TL1A expression in antigen presenting cells. In another embodiment, the present invention provides methods of treatment of inflammatory bowel disease by inhibition of IFN-gamma production in CD4+ T cells.

### Use of the TL1A Genetic Haplotype and Serologic Expression for Targeting Anti-TL1A Therapy in Patients with Inflammatory Bowel Disease

As disclosed herein, the inventors have discovered techniques for assessing the level of TL1A expression in normal subjects and patients with inflammatory bowel disease, and the significance of this level as an indicator of a specific form of inflammation. This technique can be applied to any disease in which TL1A may play a role in inflammation, such as rheumatoid arthritis, multiple sclerosis, psoriasis and/or any other chronic inflammatory disease. As described further herein, TL1A (TNF super family member 15) is a recently described TNF-like molecule that is expressed in myeloid cells and T-cells.

As further disclosed herein, the inventors have shown that there is markedly increased expression of TL1A in the mucosa of Crohn's disease patients and some patients with ulcerative colitis. Expression of this molecule has been shown to be similarly increased in the synovium of patients with rheumatoid arthritis and in lesions of patients with psoriasis. The inventors defined haplotypes of the TL1A gene and have demonstrated an association with one of these haplotypes, haplotype16 (which is dominant). When monocytes from patients expressing this haplotype are signaled via the Fc-gamma-receptor, TL1A production is high. Fc-gamma-receptor stimulated monocytes from patients expressing this haplotype have augmented T cell production of IFN-gamma. Patients with the haplotype 1, i.e., 1-1 or non-16, have very low levels of TL1A and stimulated monocytes do not augment T cell production of IFN-gamma. Another aspect of this invention is the discovery that antibodies to TL1A can attenuate chronic colitis in the DSS mouse model.

In summary, the invention relates to the fact that TL1A is an important contributor to chronic inflammation in a subset of patients with Crohn's disease who express TL1A haplotype 16 and who also express the serologic marker OmpC. Anti-TNF therapies and therapeutics designed to inhibit TL1A would be targeted to patients who have this particular haplotype and/or OmpC positive serologies. In contrast, patients who have the haplotype 1 or 1 non-16, who express low levels of TL1A are unlikely to benefit from this approach. Thus, various methods are possible where patients could be selected based upon the presence or absence of specific TL1A haplotypes and/or serologies, followed by a specific corresponding treatment. The invention represents an approach for blockage of this molecule in a patient-specific fashion.

In one embodiment, the present invention provides methods of diagnosing and/or predicting susceptibility to Crohn's Disease in an individual by determining the presence or absence of expression of TL1A haplotype 16 and/or serologic marker OmpC. In another embodiment, the present invention provides methods of prognosis of Crohn's Disease in an individual by determining the presence or absence of expression of TL1A haplotype 16 and/or serologic marker OmpC. In another embodiment, the present invention provides methods of treatment of Crohn's Disease by determining the presence or absence in the individual of expression of TL1A haplotype 16 and/or serologic marker OmpC, and then inhibiting TL1A expression.

In one embodiment, the present invention provides methods of treatment of inflammatory bowel disease by inhibition of TL1A expression by TL1A mAb. In another embodiment, the present invention provides methods of treatment of inflammatory bowel disease by inhibition of TL1A expression by TL1A mAb in individuals who express TL1A haplotype 16 and/or serologic marker OmpC. In another embodiment, the present invention provides methods of treatment of inflammatory bowel disease by using RNAi to inhibit expression of TL1A in individuals who express TL1A haplotype 16 and/or serologic marker OmpC.

In one embodiment, the present invention provides methods of treatment of inflammation by inhibition of TL1A expression in individuals who express TL1A haplotype 16 and/or serologic marker OmpC.

In one embodiment, the present invention provides methods of diagnosing and/or predicting susceptibility to Crohn's Disease in an individual by determining the presence or absence of expression of TL1A haplotype 16 in monocytes signaled via the Fc-gamma-receptor. In another embodiment, the present invention provides methods of prognosis of Crohn's Disease in an individual by determining the presence or absence of expression of TL1A haplotype 16 in monocytes signaled via the Fc-gamma-receptor. In another embodiment, the present invention provides methods of treatment of Crohn's Disease by determining the presence or absence in the individual of expression of TL1A haplotype 16 in monocytes signaled via the Fc-gamma-receptor.

### TNFSF15 Regulation of Th1 and Th17 Function

As further disclosed herein, the inventors demonstrated that TL1A (TNFSF15) is a master regulator of Th1 and Th17 function. This is a unique property of TL1A. The mechanism of TL1A blockade of mucosal inflammation is by inhibiting both Th1 and Th17 function using a single target.

In one embodiment, the present invention provides methods of diagnosing and/or predicting Th1 and/or Th17 mediated disease by determining the presence or absence of expression of TL1A. In another embodiment, the present invention provides methods of prognosis of Th1 and/or Th17 mediated disease in an individual by determining the presence or absence of expression of TL1A. In another embodiment, the present invention provides methods of treatment of Th1 and/or Th17 mediated disease by inhibiting expression of TL1A.

### Variety of Methods and Materials

A variety of methods can be used to determine the presence or absence of a variant allele or haplotype. As an example, enzymatic amplification of nucleic acid from an individual may be used to obtain nucleic acid for subsequent analysis. The presence or absence of a variant allele or haplotype may also be determined directly from the individual's nucleic acid without enzymatic amplification.

Analysis of the nucleic acid from an individual, whether amplified or not, may be performed using any of various techniques. Useful techniques include, without limitation, polymerase chain reaction based analysis, sequence analysis and electrophoretic analysis. As used herein, the term “nucleic acid” means a polynucleotide such as a single or double-stranded DNA or RNA molecule including, for example, genomic DNA, cDNA and mRNA. The term nucleic acid encompasses nucleic acid molecules of both natural and synthetic origin as well as molecules of linear, circular or branched configuration representing either the sense or antisense strand, or both, of a native nucleic acid molecule.

The presence or absence of a variant allele or haplotype may involve amplification of an individual's nucleic acid by the polymerase chain reaction. Use of the polymerase chain reaction for the amplification of nucleic acids is well known in the art (see, for example, Mullis et al. (Eds.), The Polymerase Chain Reaction, Birkhauser, Boston, (1994)).

A TaqmanB allelic discrimination assay available from Applied Biosystems may be useful for determining the presence or absence of a TL1A variant allele. In a TaqmanB allelic discrimination assay, a specific, fluorescent, dye-labeled probe for each allele is constructed. The probes contain different fluorescent reporter dyes such as FAM and VICTM to differentiate the amplification of each allele. In addition, each probe has a quencher dye at one end which quenches fluorescence by fluorescence resonant energy transfer (FRET). During PCR, each probe anneals specifically to complementary sequences in the nucleic acid from the individual. The 5′ nuclease activity of Taq polymerase is used to cleave only probe that hybridize to the allele. Cleavage separates the reporter dye from the quencher dye, resulting in increased fluorescence by the reporter dye. Thus, the fluorescence signal generated by PCR amplification indicates which alleles are present in the sample. Mismatches between a probe and allele reduce the efficiency of both probe hybridization and cleavage by Taq polymerase, resulting in little to no fluorescent signal. Improved specificity in allelic discrimination assays can be achieved by conjugating a DNA minor grove binder (MGB) group to a DNA probe as described, for example, in Kutyavin et al., “3′-minor groove binder-DNA probes increase sequence specificity at PCR extension temperature, “Nucleic Acids Research 28:655-661 (2000)). Minor grove binders include, but are not limited to, compounds such as dihydrocyclopyrroloindole tripeptide (DPI).

Sequence analysis also may also be useful for determining the presence or absence of a TL1A variant allele or haplotype.

Restriction fragment length polymorphism (RFLP) analysis may also be useful for determining the presence or absence of a particular allele (Jarcho et al. in Dracopoli et al., Current Protocols in Human Genetics pages 2.7.1-2.7.5, John Wiley & Sons, New York; Innis et al., (Ed.), PCR Protocols, San Diego: Academic Press, Inc. (1990)). As used herein, restriction fragment length polymorphism analysis is any method for distinguishing genetic polymorphisms using a restriction enzyme, which is an endonuclease that catalyzes the degradation of nucleic acid and recognizes a specific base sequence, generally a palindrome or inverted repeat. One skilled in the art understands that the use of RFLP analysis depends upon an enzyme that can differentiate two alleles at a polymorphic site.

Allele-specific oligonucleotide hybridization may also be used to detect a disease-predisposing allele. Allele-specific oligonucleotide hybridization is based on the use of a labeled oligonucleotide probe having a sequence perfectly complementary, for example, to the sequence encompassing a disease-predisposing allele. Under appropriate conditions, the allele-specific probe hybridizes to a nucleic acid containing the disease-predisposing allele but does not hybridize to the one or more other alleles, which have one or more nucleotide mismatches as compared to the probe. If desired, a second allele-specific oligonucleotide probe that matches an alternate allele also can be used. Similarly, the technique of allele-specific oligonucleotide amplification can be used to selectively amplify, for example, a disease-predisposing allele by using an allele-specific oligonucleotide primer that is perfectly complementary to the nucleotide sequence of the disease-predisposing allele but which has one or more mismatches as compared to other alleles (Mullis et al., supra, (1994)). One skilled in the art understands that the one or more nucleotide mismatches that distinguish between the disease-predisposing allele and one or more other alleles are preferably located in the center of an allele-specific oligonucleotide primer to be used in allele-specific oligonucleotide hybridization. In contrast, an allele-specific oligonucleotide primer to be used in PCR amplification preferably contains the one or more nucleotide mismatches that distinguish between the disease-associated and other alleles at the 3′ end of the primer.

A heteroduplex mobility assay (HMA) is another well known assay that may be used to detect a SNP or a haplotype. HMA is useful for detecting the presence of a polymorphic sequence since a DNA duplex carrying a mismatch has reduced mobility in a polyacrylamide gel compared to the mobility of a perfectly base-paired duplex (Delwart et al., Science 262:1257-1261 (1993); White et al., Genomics 12:301-306 (1992)).

The technique of single strand conformational, polymorphism (SSCP) also may be used to detect the presence or absence of a SNP and/or a haplotype (see Hayashi, K., Methods Applic. 1:34-38 (1991)). This technique can be used to detect mutations based on differences in the secondary structure of single-strand DNA that produce an altered electrophoretic mobility upon non-denaturing gel electrophoresis. Polymorphic fragments are detected by comparison of the electrophoretic pattern of the test fragment to corresponding standard fragments containing known alleles.

Denaturing gradient gel electrophoresis (DGGE) also may be used to detect a SNP and/or a haplotype. In DGGE, double-stranded DNA is electrophoresed in a gel containing an increasing concentration of denaturant; double-stranded fragments made up of mismatched alleles have segments that melt more rapidly, causing such fragments to migrate differently as compared to perfectly complementary sequences (Sheffield et al., “Identifying DNA Polymorphisms by Denaturing Gradient Gel Electrophoresis” in Innis et al., supra, 1990).

Other molecular methods useful for determining the presence or absence of a SNP and/or a haplotype are known in the art and useful in the methods of the invention. Other well-known approaches for determining the presence or absence of a SNP and/or a haplotype include automated sequencing and RNAase mismatch techniques (Winter et al., Proc. Natl. Acad. Sci. 82:7575-7579 (1985)). Furthermore, one skilled in the art understands that, where the presence or absence of multiple alleles or haplotype(s) is to be determined, individual alleles can be detected by any combination of molecular methods. See, in general, Birren et al. (Eds.) Genome Analysis: A Laboratory Manual Volume 1 (Analyzing DNA) New York, Cold Spring Harbor Laboratory Press (1997). In addition, one skilled in the art understands that multiple alleles can be detected in individual reactions or in a single reaction (a “multiplex” assay). In view of the above, one skilled in the art realizes that the methods of the present invention for diagnosing or predicting susceptibility to or protection against CD in an individual may be practiced using one or any combination of the well known assays described above or another art-recognized genetic assay.

One skilled in the art will recognize many methods and materials similar or equivalent to those described herein, which could be used in the practice of the present invention. Indeed, the present invention is in no way limited to the methods and materials described. For purposes of the present invention, the following terms are defined below.

### EXAMPLES

The following examples are provided to better illustrate the claimed invention and are not to be interpreted as limiting the scope of the invention. To the extent that specific materials are mentioned, it is merely for purposes of illustration and is not intended to limit the invention. One skilled in the art may develop equivalent means or reactants without the exercise of inventive capacity and without departing from the scope of the invention.

### Example 1

### Novel Association Between TNFSF15 and Disease Phenotype

DNA was collected from 250 well characterized pediatric CD cases. Genotyping (TaqmanMGB) was performed for 3 CD-associated variants of NOD2, single nucleotide polymorphisms (SNPs) 8, 12, and 13 and for 5 polymorphisms of TNFSF15 gene, rs3810936, rs6478108, rs6478109, rs7848647, rs7869487. Haplotypes were constructed using PHASE 2.1.1. Phenotypes were determined by chart review blinded to genotype. Associations between candidate genes and disease phenotype (location and complicated behavior [internal penetrating and/or stricturing disease (IP/S)]) were determined.

NOD2 variants were found in 32% of children and the carrier frequency of common alleles for TNFSF15 ranged from 90%-93%. The frequencies of the 2 most common TNFSF15 haplotypes, A and B, were 66% and 21%, respectively. NOD2 was not associated with disease location or behavior. In contrast, haplotype A of TNFSF15 was associated with small bowel (SB) (p=0.02) and IP/S (p=0.05). Carriage of the common allele of rs6478109 (−358) had the strongest association with both SB (p<0.001) and IP/S (p<0.02). There was no interaction between NOD2 and TNFSF15.

Thus, there is an association between TNFSF15 and disease phenotype in CD patients. Carriage of the common haplotype was associated with complicated small bowel disease. Given its location, SNP rs6478109 plays a role in gene expression and this in turn modifies disease phenotype.

### Example 2

### TNFSF15 is an Ethnic Specific Gene

Five SNPs that comprise two common haplotypes were genotyped in 572 Caucasian patients with Crohn's disease (CD), 377 Caucasian patients with UlcerativeColitis (UC) and 216 ethnically-matched healthy controls, including both Jews and non-Jews.

The previously reported ‘risk’ haplotype was not associated with CD or UC (88.3% in CD cases vs. 88.9% in controls, p=0.81; 88.9% in UC cases vs. 88.9% in controls, p=0.99). The inventors did however observe an increased frequency of the “protective” haplotype in Non-Jewish controls for both CD and UC (39.3% CD cases vs. 49.1% controls, p=0.03; 37.6% UC cases vs. 49.1% controls, p=0.02) with no such effect observed in the Jewish samples. There was an interactive effect between ethnicity and the protective haplotype in CD (p=0.04).

The inventors observed a protective haplotype, consisting of the minor alleles for all five markers, to have a higher frequency in the non-Jewish controls than in CD and UC. Of further interest, the haplotype frequency was in the opposite direction, in the Jewish case-control panels (both CD and UC), leading the inventors to conclude (1) TNFSF15 is indeed an IBD susceptibility gene, and (2) the disease susceptibility is ethnic specific.

### Example 3

### TNFSF15 is an Ethnic Specific Gene: Tables

Five genotyped SNPs formed three common haplotypes in our population (A, B and C). The haplotypes and their frequencies are shown in Tables 1 and 2. Overall, haplotype A had a carrier frequency in controls of 88.9%, haplotype B 43.5%, and haplotype C 6.9%. However, the overall result masked distinct ethnic differences. In non-Jews, the haplotype frequencies in controls were 88.3%, 49.1% and 5.9% respectively, while in Jews they were 91.1%, 22.2% and 11.1%.

### Tables 1 and 2.

TABLE 1
TNFSF16 haplotype Carrier association with CD

| | overall | | | | Non-Jewish | | | | Non-Jewish | | | |
|---|---|---|---|---|---|---|---|---|---|---|---|---|
| | Cases | Controls | | OR | Cases | Controls | | | Cases | Controls | | OR |
| Haplotype | (n = 572) | (n = 216) | P | (95% CI) | (n = 328) | (n = 171) | P | OR | (n = 244) | (n = 45) | P | (95% CI) |
| Haplo. A | 88.3% | 88.9% | 0.81 | 0.94 | 88.1% | 88.3% | 0.95 | 0.98 | 88.5% | 91.1% | 0.80 | 0.75 |
| | | | | (0.57, 1.54) | | | | (0.55, 1.74) | | | | (0.18, 2.32) |
| Haplo. B | 36.0% | 43.5% | 0.05 | 0.73 | 39.3% | 49.1% | 0.03 | 0.57 | 31.16% | 22.2% | 0.21 | 1.61 |
| | | | | (0.53, 1.00) | | | | (0.46, 0.97) | | | | (0.76, 3.43) |
| Haplo. C | 5.6% | 6.9% | 0.47 | 0.79 | 5.2% | 5.9% | 0.75 | 0.88 | 6.1% | 11.1% | 0.21 | 0.52 |
| | | | | (0.42, 1.50) | | | | (0.39, 1.97) | | | | (0.17, 1.95) |

TABLE 2
TNFSF15 haplotype Carrier association with UC

| | overall | | | | Non-Jewish | | | | Jewish | | | |
|---|---|---|---|---|---|---|---|---|---|---|---|---|
| | Cases | Controls | | OR | Cases | Controls | | | Cases | Controls | | OR |
| Haplotype | (n = 377) | (n = 216) | P | (95% CI) | (n = 234) | (n = 171) | P | OR | (n = 143) | (n = 45) | P | (95% CI) |
| Haplo. A | 88.9% | 88.9% | 0.99 | 1.00 | 89.7% | 88.3% | 0.65 | 1.16 | 87.4% | 91.1% | 0.50 | 0.68 |
| | | | | (0.58, 1.70) | | | | (0.62, 2.17) | | | | (0.22, 2.12) |
| Haplo. B | 33.2% | 43.5% | 0.01 | 0.64 | 37.6% | 49.1% | 0.02 | 0.62 | 25.9% | 22.2% | 0.62 | 1.22 |
| | | | | (0.46, 0.91) | | | | (0.42, 0.93) | | | | (0.55, 2.71) |
| Haplo. C | 4.2% | 6.9% | 0.15 | 0.59 | 3.4% | 5.9% | 0.24 | 0.57 | 5.6% | 11.1% | 0.31 | 0.47 |
| | | | | (0.29, 1.23) | | | | (0.22, 1.47) | | | | (0.15, 1.53) |

When the results are separated by ethnicity into non-Jewish and Jewish subgroups, haplotype B carriers, consisting of the rare allele for each marker, were found to be protective against CD, but specifically only in non-Jews (39.3% in CD vs 49.1% in controls, OR: 0.67 CI: 0.46-0.97, p=0.03) In contrast, Haplotype B frequencies had an opposite trend in the Jewish population, with 31.6% in the CD cases versus 22.2% in controls; this difference was not significant (p=0.21). The ethnic distinction was further verified through logistic regression, in which an interactive effect between ethnicity and haplotype B in CD was detected (p=0.04). The same haplotype analysis was performed on the UC cohort. The combined UC cohort (including both Jewish and non-Jewish panels) did show an association between haplotype B and UC as shown in Table 2 (p=0.01, OR: 0.64 CI: 0.46-0.91.) However, the association was clarified when results were separated by ethnicity, with the non-Jewish population having a significant association with haplotype B (37.6% vs 49.1%, OR: 0.62 CI: 0.42-0.93, p=0.02.) In contrast, the Jewish UC cohort was opposite in trend in haplotype B frequency as well, with haplotype B being more frequent in cases (25.9%) than in controls (22.2%), although this difference was not significant. There was a trend toward interaction between ethnicity and haplotype B in UC (P=0.1); this did not attain statistical significance.

The results demonstrate an association between the low-risk haplotype (B) and both CD and UC in a non-Jewish Caucasian population. Also identified is a distinct ethnic relationship, in that the result is confined to non-Jews. This confirms and clarifies that haplotype B is protective against IBD in a Caucasian population, but it is specifically in a non-Jewish Caucasian population. The frequency trend of haplotype B was in fact in the opposite direction in the Jewish CD and UC panels compared with the direction seen in the non-Jewish panels and Caucasian cohorts. This protective haplotype association with both UC and with CD in the non-Jewish cohort, demonstrates that this gene is not CD or UC specific, but is in fact an IBD gene.

### Example 4

### TNFSF15 is an Ethnic Specific Gene: Patients

1165 Caucasian subjects were ascertained from Cedars-Sinai Medical Center. The cohort consisted of 572 CD patients, 377 ulcerative colitis patients and 216 ethnically-matched controls. Part of this cohort has been studied previously 31-33. The Institutional Review Board at Cedars-Sinai Medical Center approved the study protocol, and informed consent was obtained from all study subjects. The diagnosis of Crohn's disease or ulcerative colitis was based on standard criteria including clinical, endoscopic, radiographic, and histopathological criteria 31, 32, 34-36. Patients with indeterminate colitis were excluded. Also, cohort was evaluated on ethnicity, Jewish or non-Jewish. Jewish (J) was defined as those individuals with at least one out of four grandparents of Ashkenazi Jewish origin 37, 38.

### Example 5

### TNFSF15 is an Ethnic Specific Gene: Genotyping

DNA was extracted from the peripheral whole blood collected using PureGene DNA Isolation kit (Gentra Systems; Minneapolis, Minn.). Five single nucleotide polymorphisms (SNP) previously found to be associated with CD and/or UC in a Japanese cohort (rs3810936, rs6478108, rs6478109, rs7848647, rs7869487) were tested 20. These SNPs were used to construct 2 common haplotypes previously seen in both a Japanese and a Caucasian population 20. Probes and primers were designed for the TaqMan MGB allelic discrimination method (Applied Biosystems, Foster City, Calif.). The fluorescent amplifications were measured using the ABI Prism 7000 Sequence Detection System.

### Example 6

### TNFSF15 is an Ethnic Specific Gene: Statistical Analysis

None of the markers exhibited significant deviation from the Hardy-Weinberg equilibrium in the sample set (p=0.05 level). Genotype distributions of individual markers were compared between cases and controls. Individual haplotypes were estimated by using Bayesian statistical method as implemented in the PHASE software (v2.0) 39, 40. Chi-square tests were used to test associations between the haplotypes and disease status. All analyses were done in total sample first, and then separated in Jewish and non-Jewish population. Multiple logistic regression was used to test the interactive effect between ethnicity and haplotype in the total sample. Statistical analysis was conducted by SAS software (SAS Institute; Cary, N.C.).

### Example 7

### The T Cell Co-Stimulator TL 1A is Induced by Fcgamma Receptor Signaling in Monocytes and Dendritic Cells

To determine whether professional T cell activating cells can express TL1A, fresh blood monocytes and monocyte-derived dendritic cells were stimulated with various activating ligands, including TLR agonists, IFN-gamma, and immune complexes. Fc-gamma-R stimulation strongly induced TL1A mRNA in both cell types, which correlated with the detection of TL1A on the cell surface and in cell culture media. TLR agonists capable of inducing IL-6 and TNF-α in monocytes and dendritic cells did not induce surface nor soluble TL1A. TL1A production in monocytes is demonstrated to lead to enhancement of T cell responses. The induction of TL1A on antigen presenting cells via specific pathway stimulation demonstrates a role for TL1A in Th1 responses to pathogens, and in CD.

### Example 8

### The T Cell Co-Stimulator TL1A is Induced by Fc-Gamma-Receptor Signaling in Monocytes and Dendritic Cells: Fc-Gamma-R Signaling Induces TL1A mRNA Expression in Monocytes and Monocyte Derived Dendritic Cells

The inventors examined whether endogenous TL1A was induced in antigen presenting cells, and, therefore, would be available to co-stimulate T cells in vivo. In order to determine which stimulus modalities might induce TL1A in monocytes and dendritic cells, the inventors stimulated cells for up to 24 h with each of several ligands known to activate these cell types, i.e., the TLR2 agonist Pam3CSK4, the TLR4 ligand LPS, CBir1 flagellin which is a ligand of TR5, IFN-gamma, and plate-bound cross-linked human IgG (plate-IC). TL1A transcript was quantified by real-time PCR using a primer/probe set specific for exon 1 and 2 of the full-length transcript. The most potent inducer for both cell types was plate IC (p<0.001). TL1A mRNA expression was expressed as % of Beta-actin. TL1A mRNA peaked by 6 h in DC, but continued to increase over 12-18 h in monocytes. In experiments in which mRNA was quantified after 6 h of stimulation and expressed as a percentage of Beta-Actin, the TLR ligands, LPS and Pam3CSK4 induced very low levels of TL1A transcript in monocytes. CBir1 flagellin and IFN-gamma were ineffective inducers in both cell types. The inventors could not detect TL1A mRNA upregulation in response to IL-1 Beta or TNF-α in human monocytes, suggesting distinct cell-type specific signaling pathways.

### Example 9

### The T Cell Co-Stimulator TL1A is Induced by Fc-Gamma-Receptor Signaling in Monocytes and Dendritic Cells: Induction of Soluble TL1A from Monocytes and Monocyte Derived Dendritic Cells by Fc-Gamma-R but not TLR Ligands, Nor IFN-Gamma Receptor Agonist

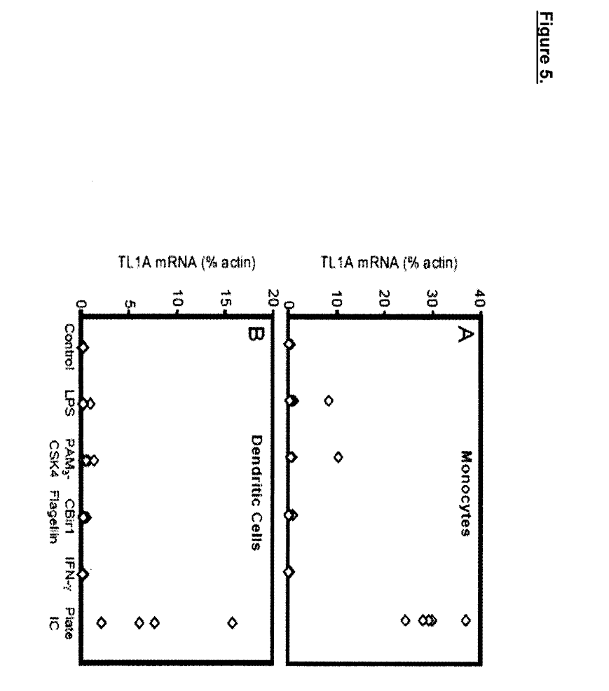

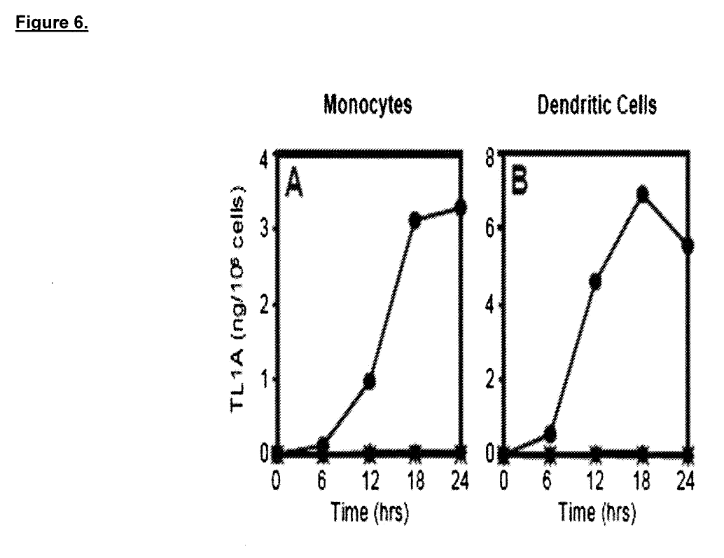

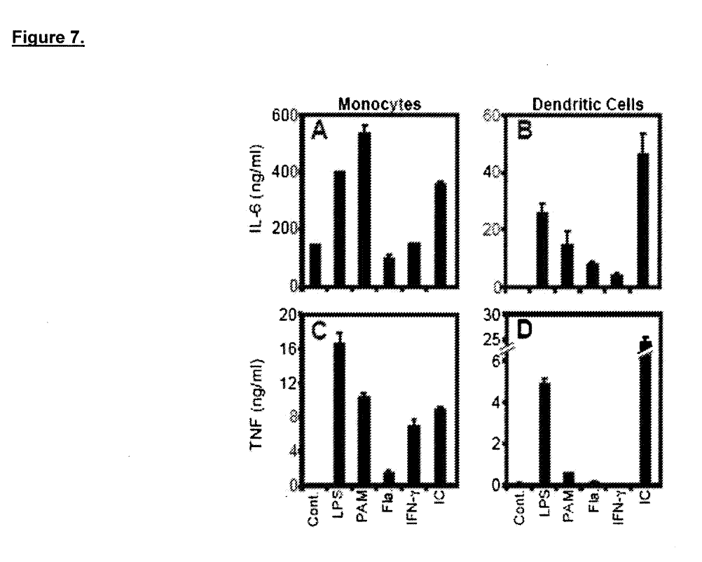

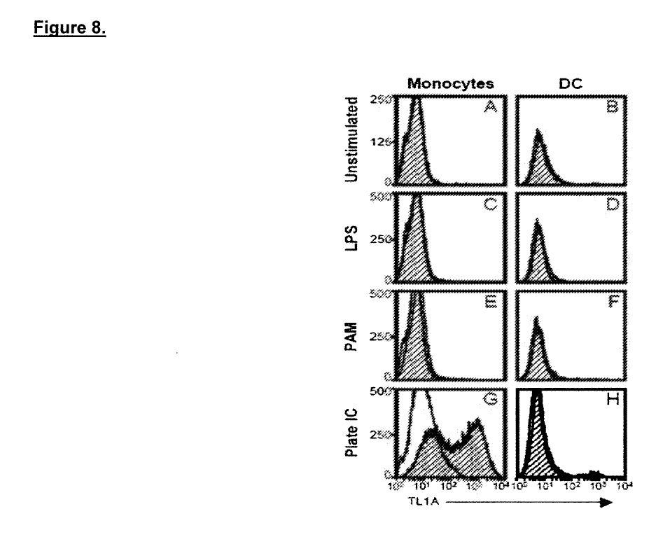

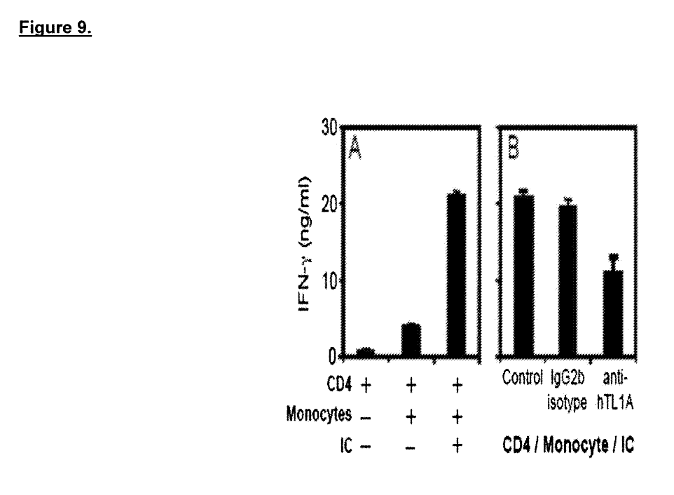

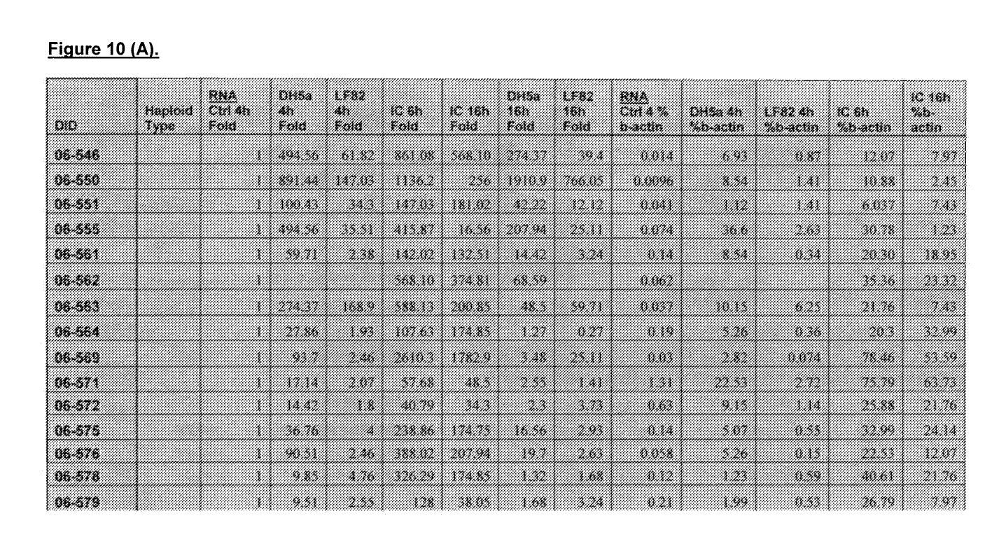

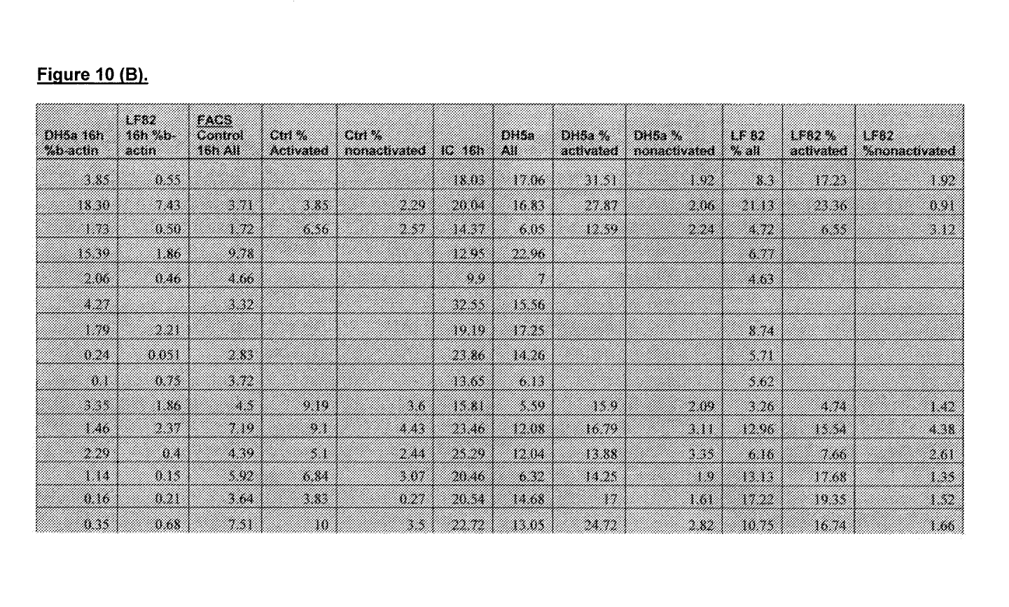

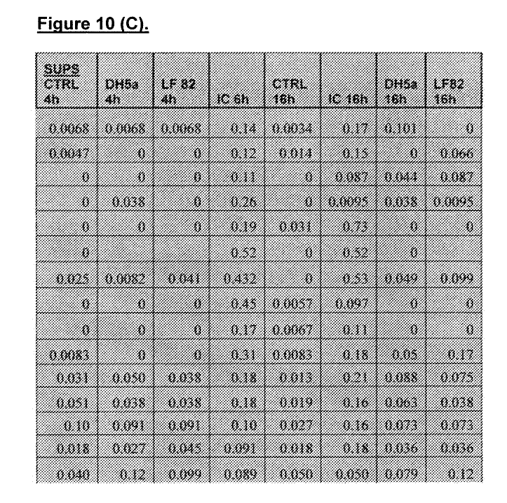

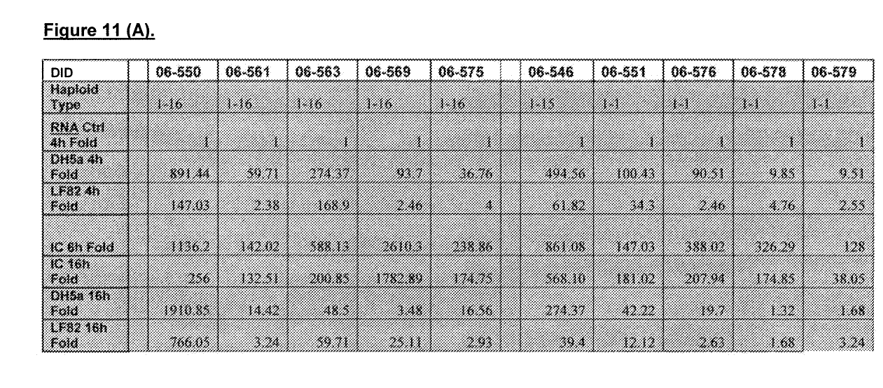

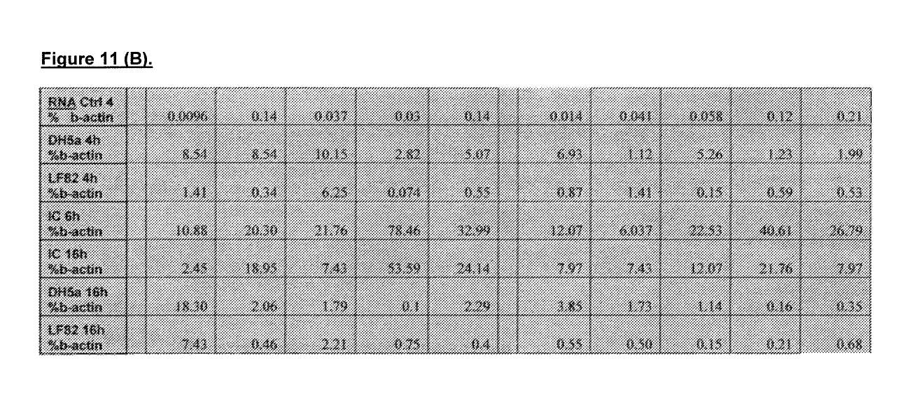

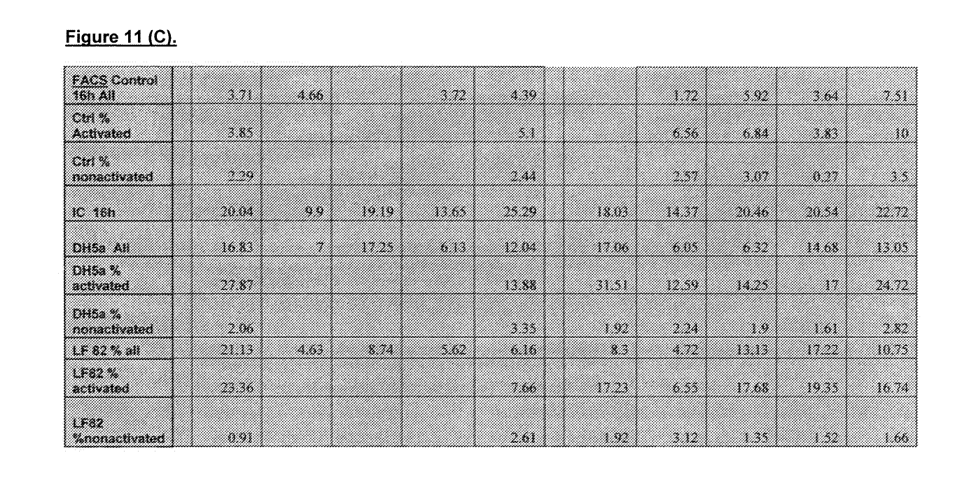

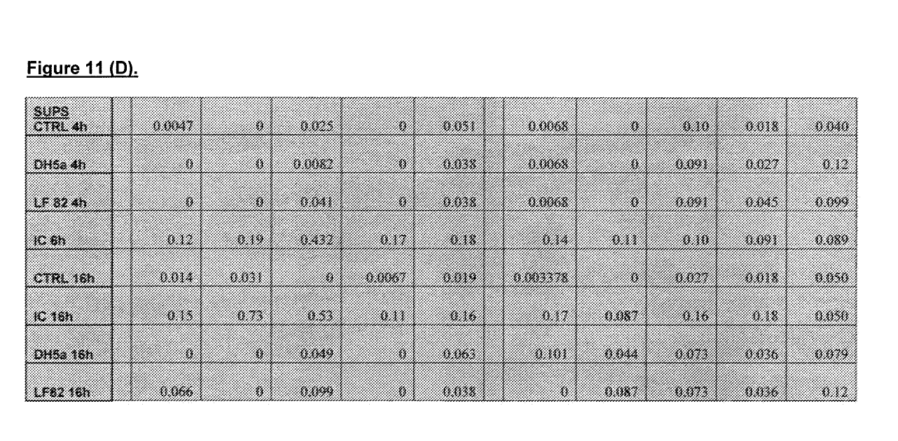

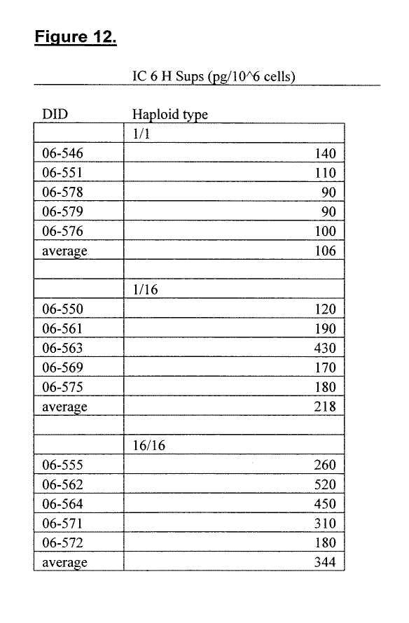

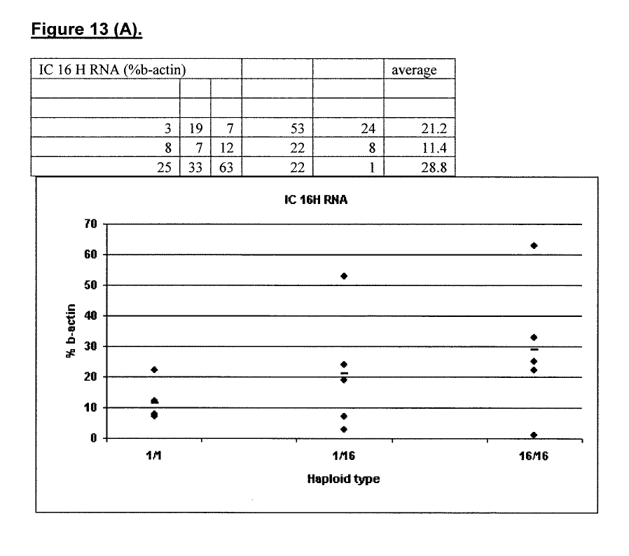

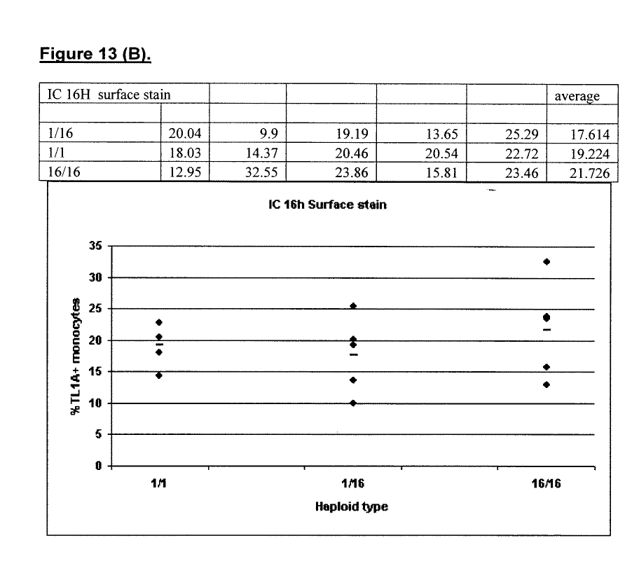

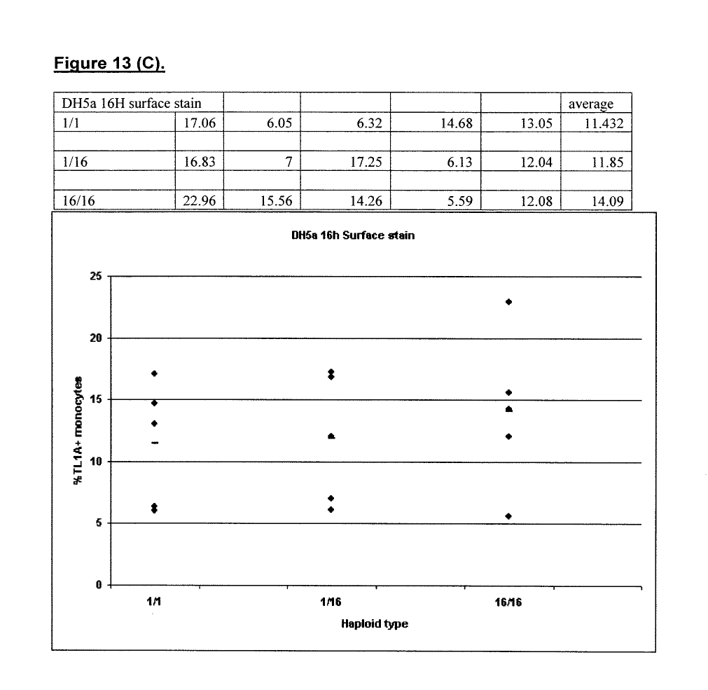

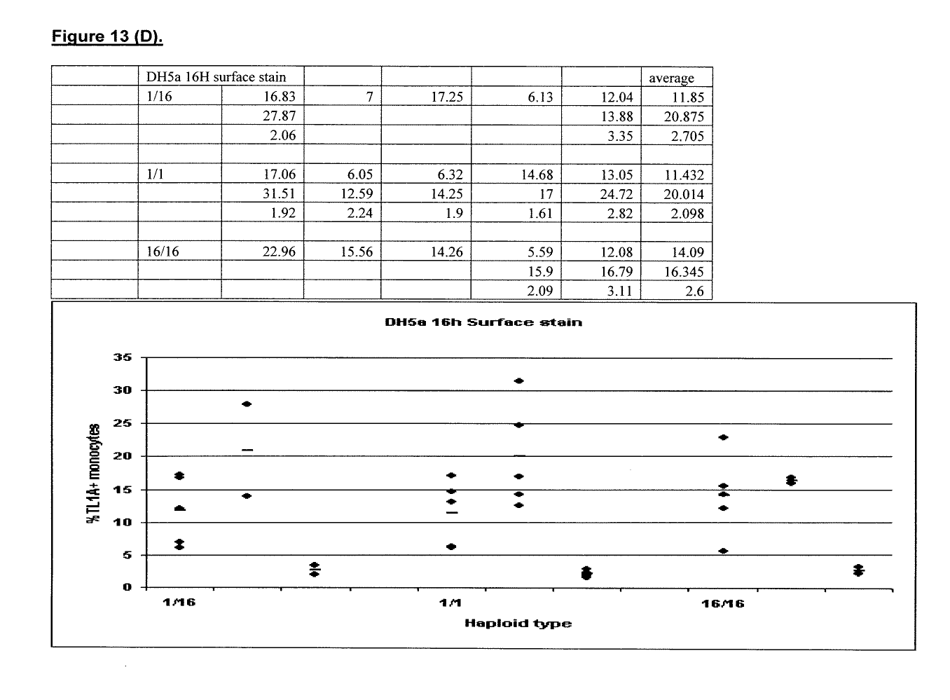

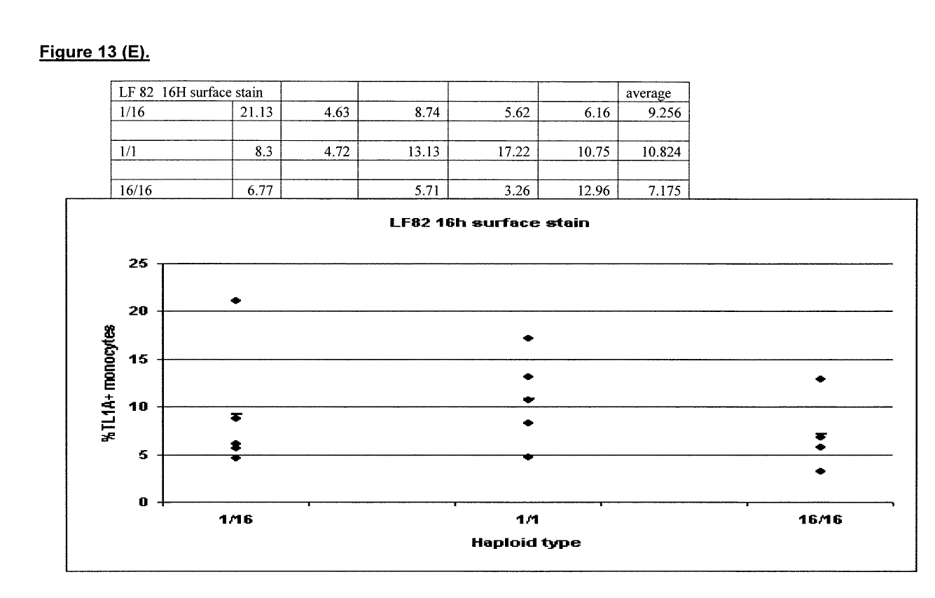

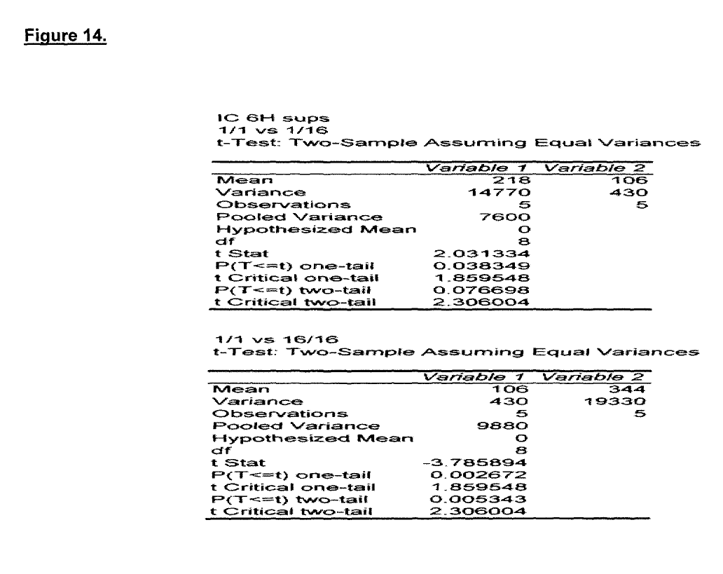

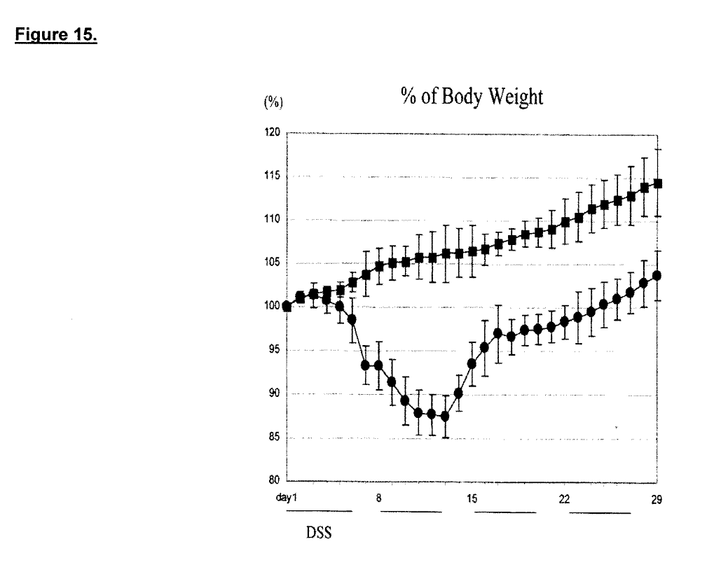

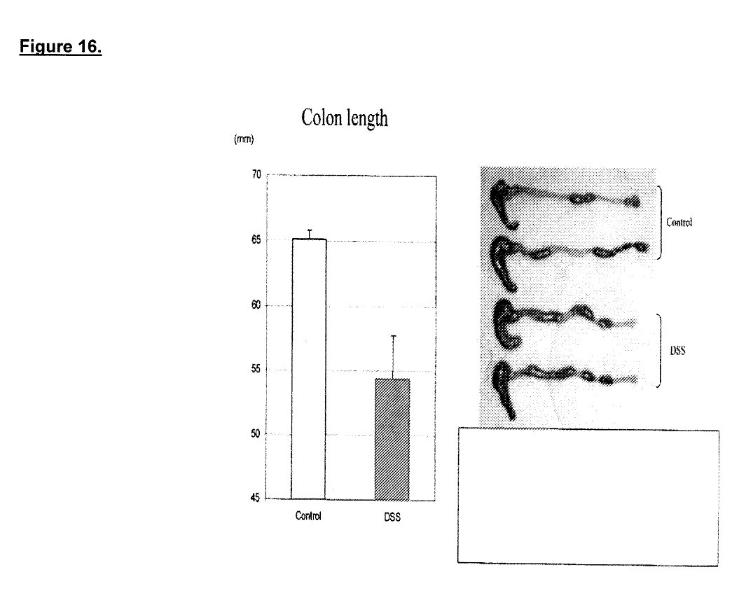

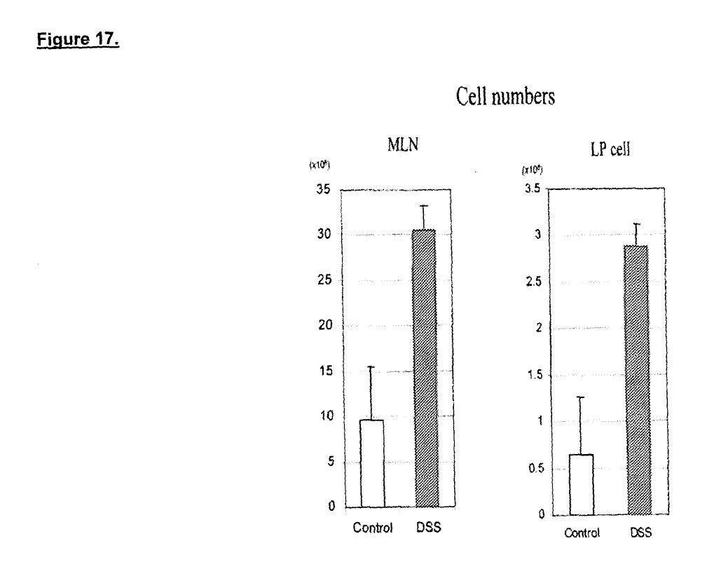

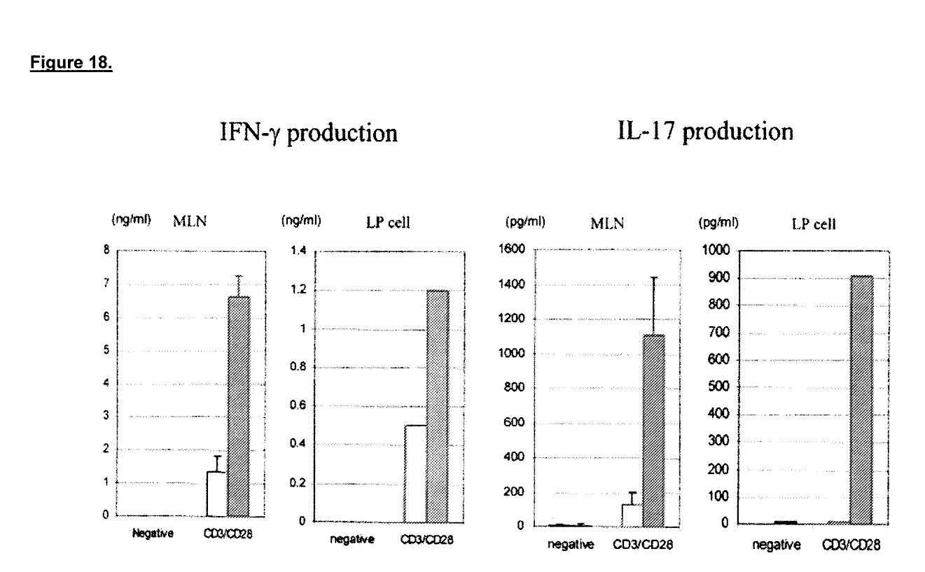

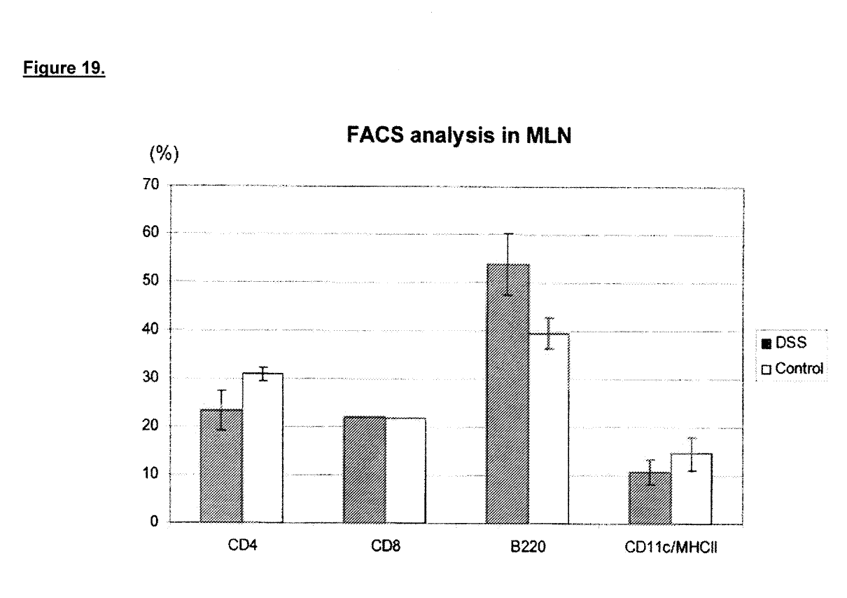

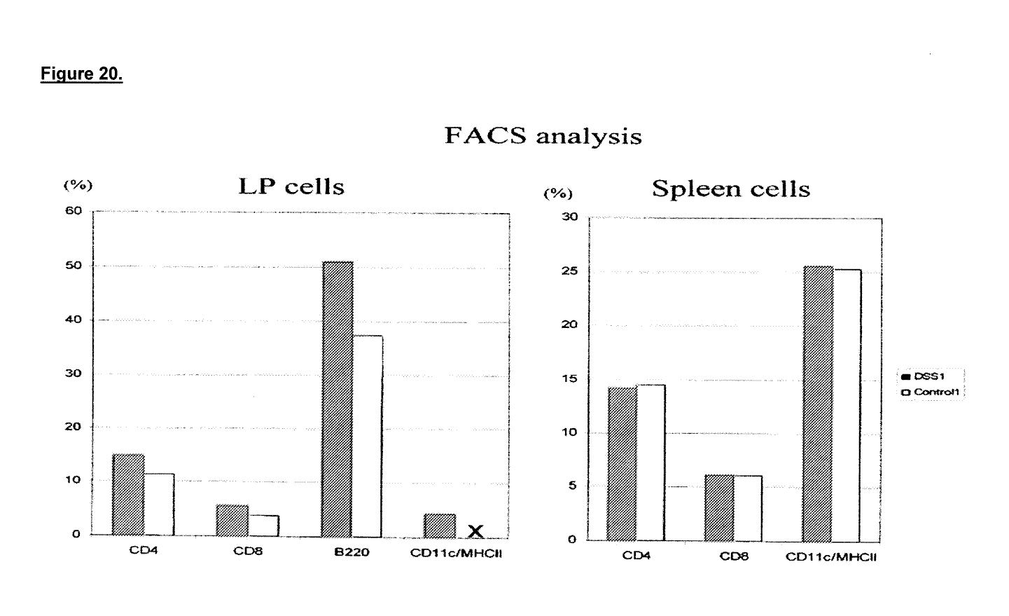

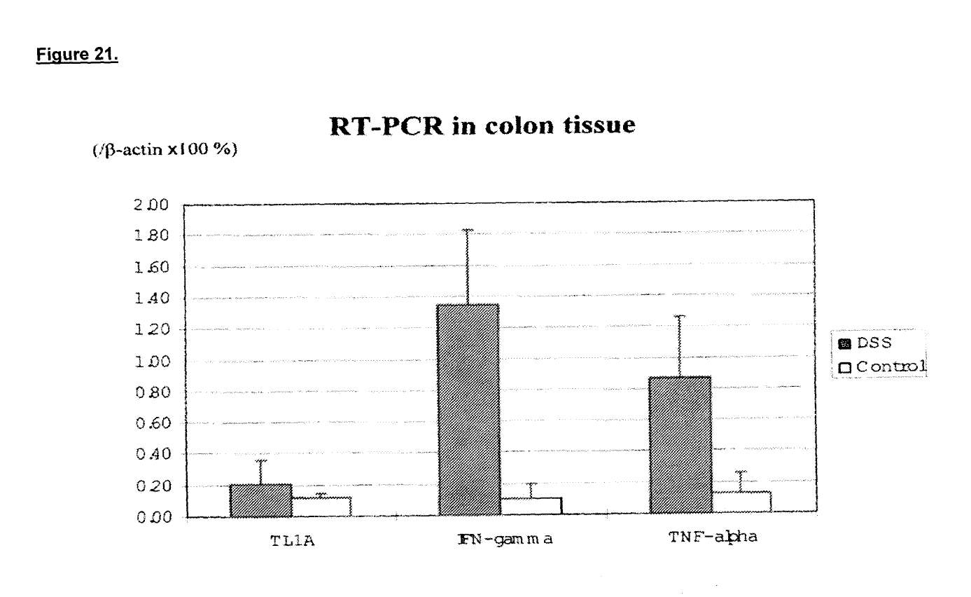

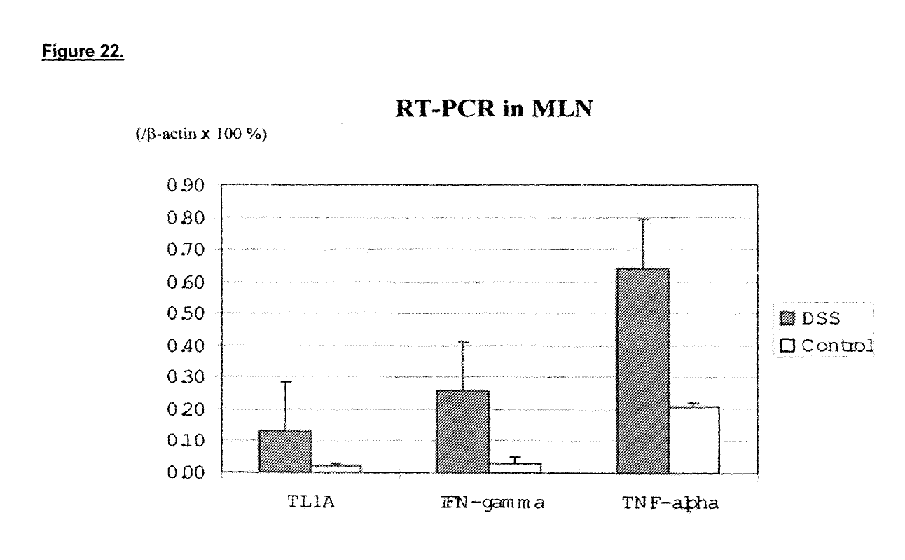

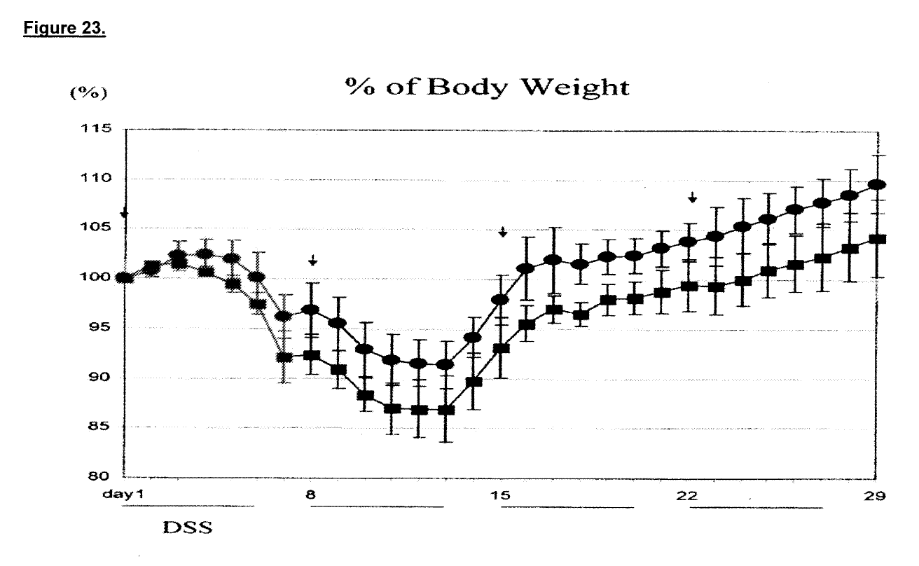

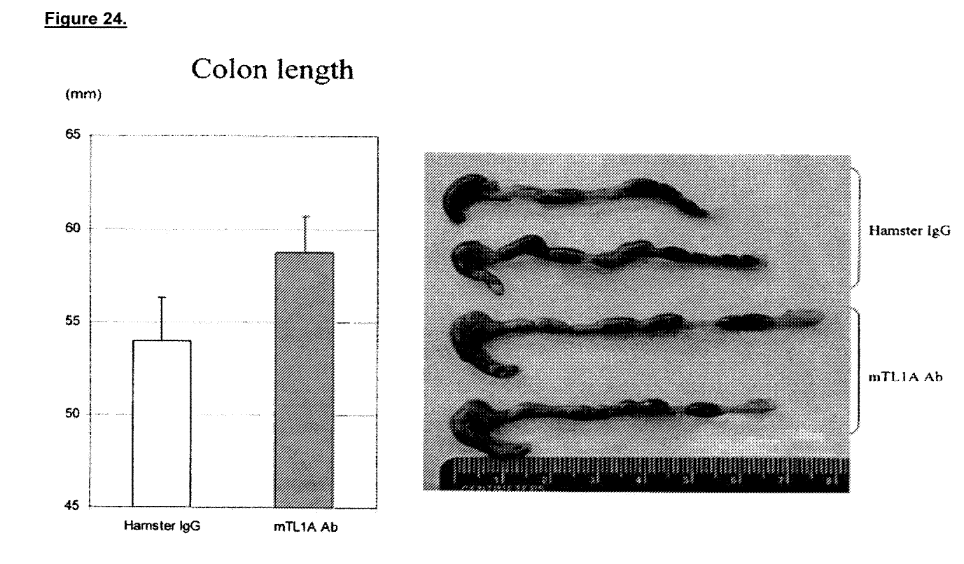

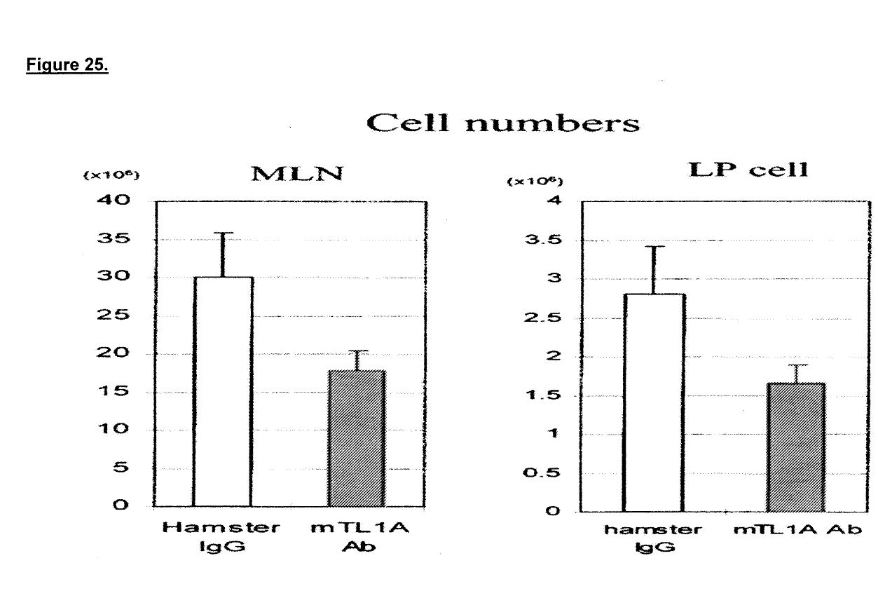

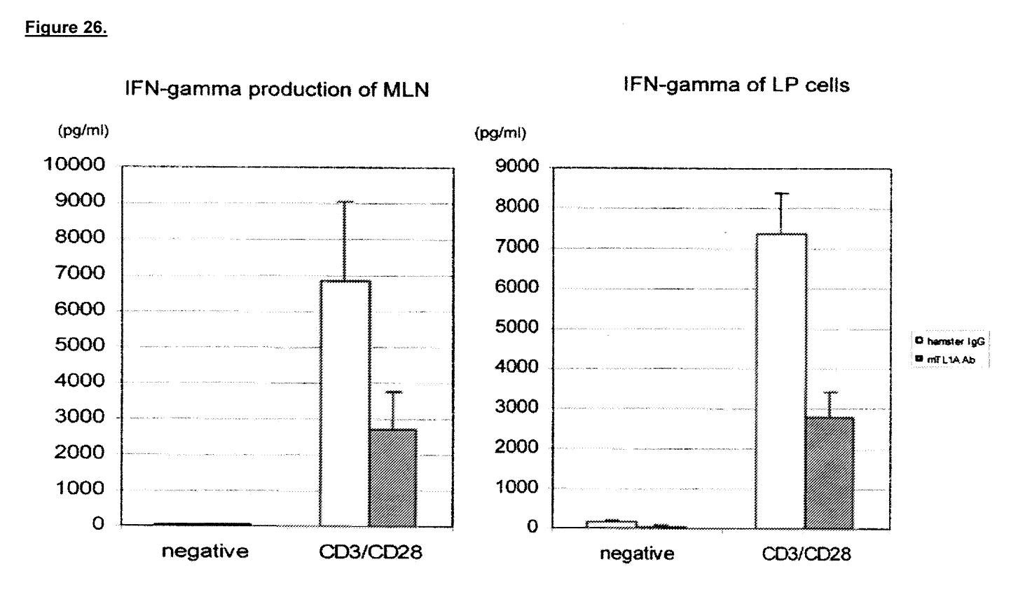

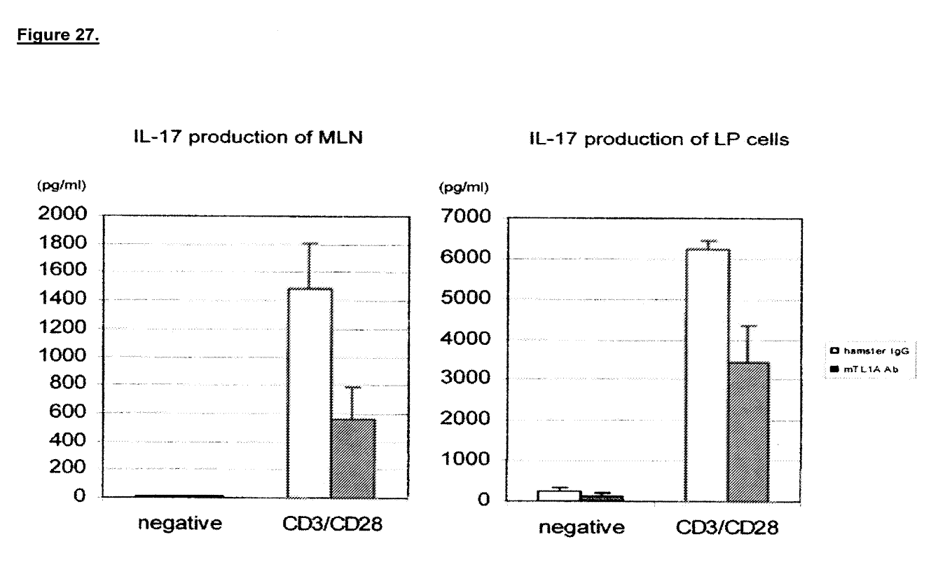

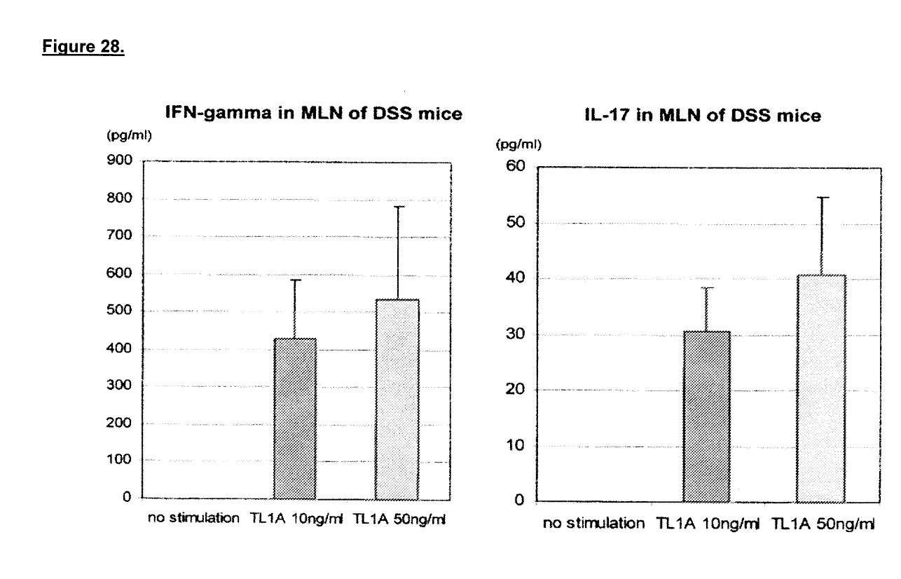

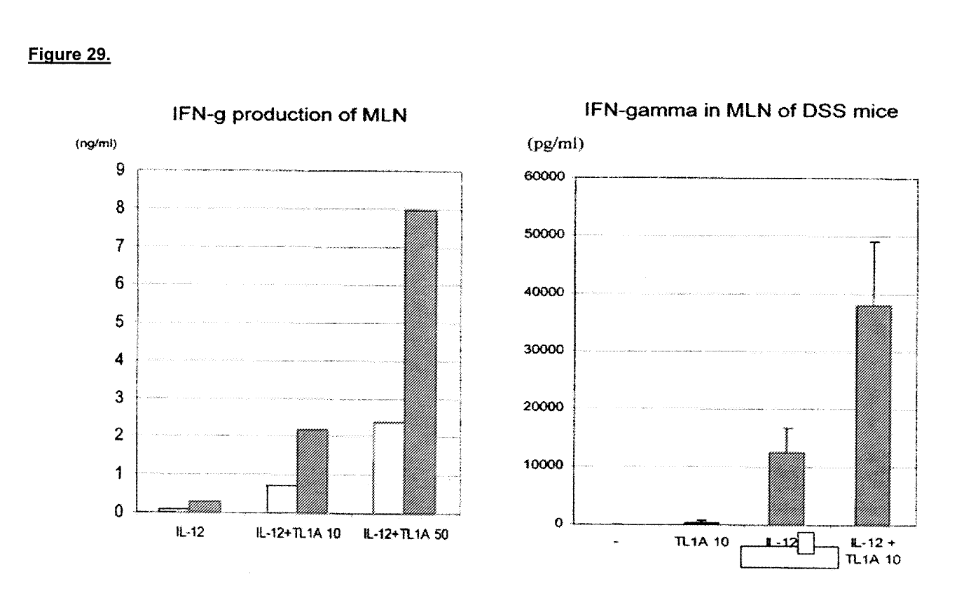

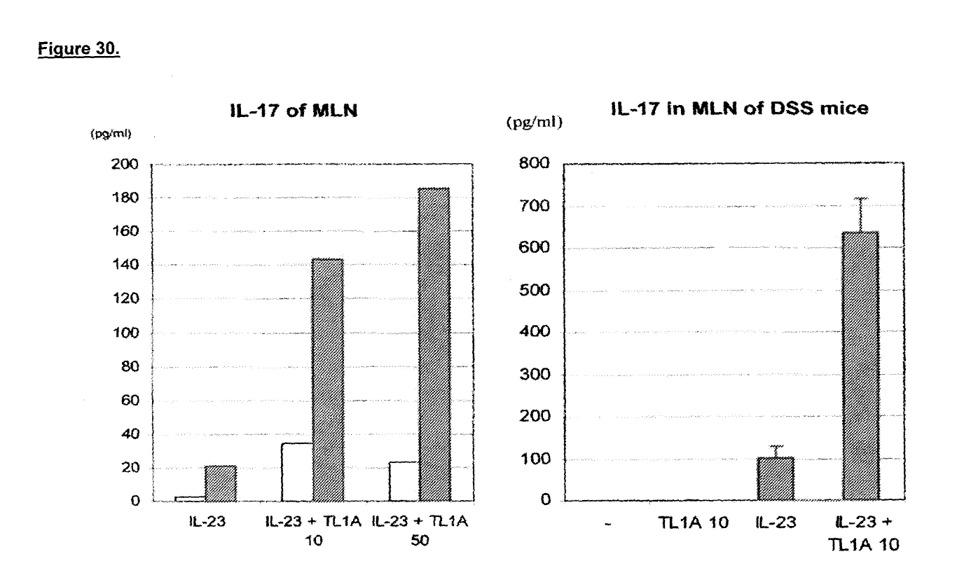

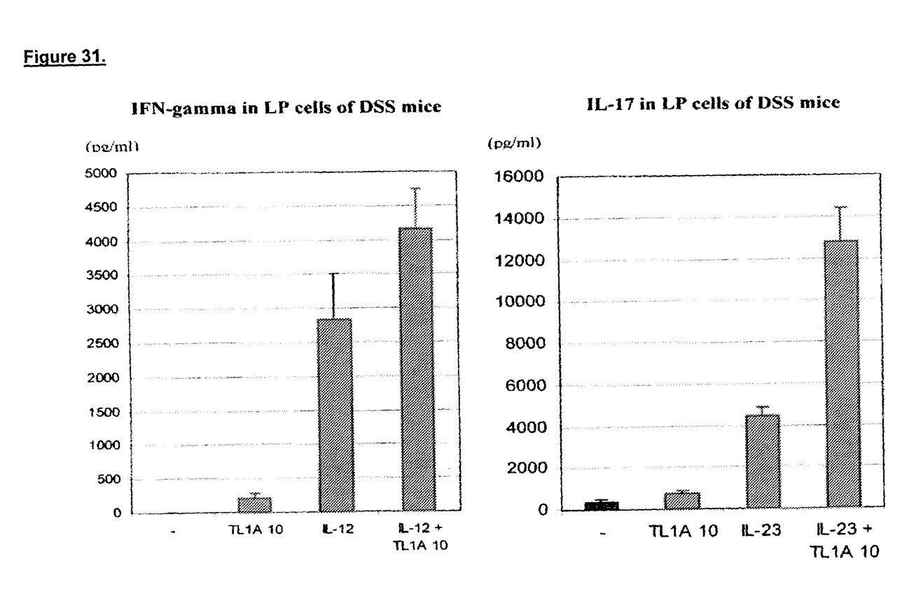

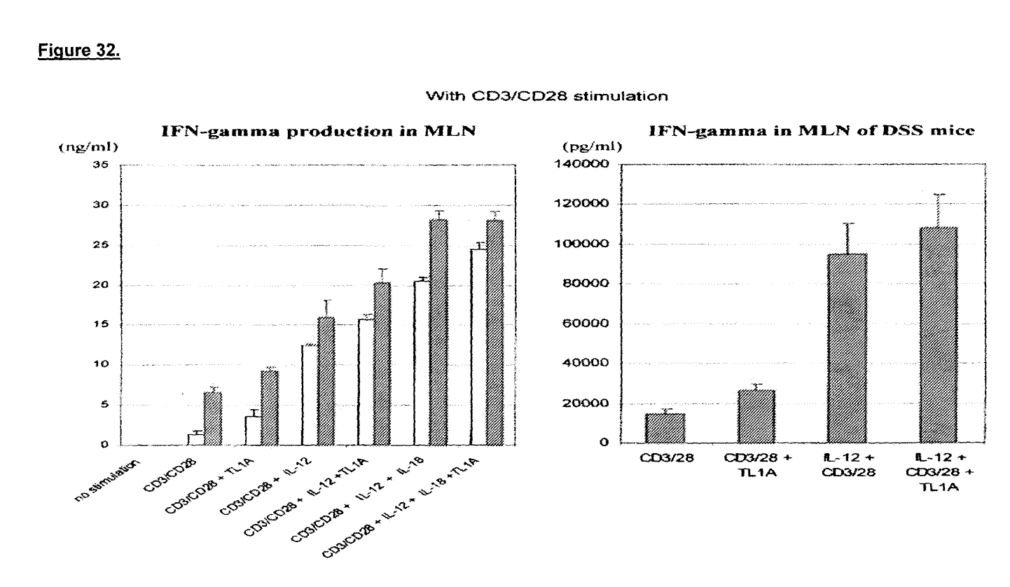

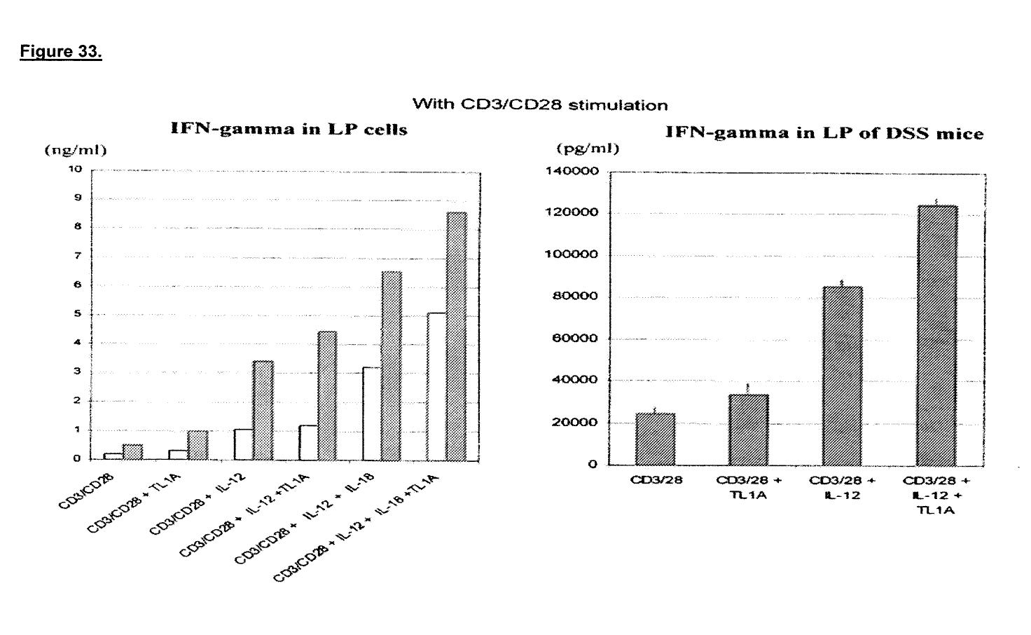

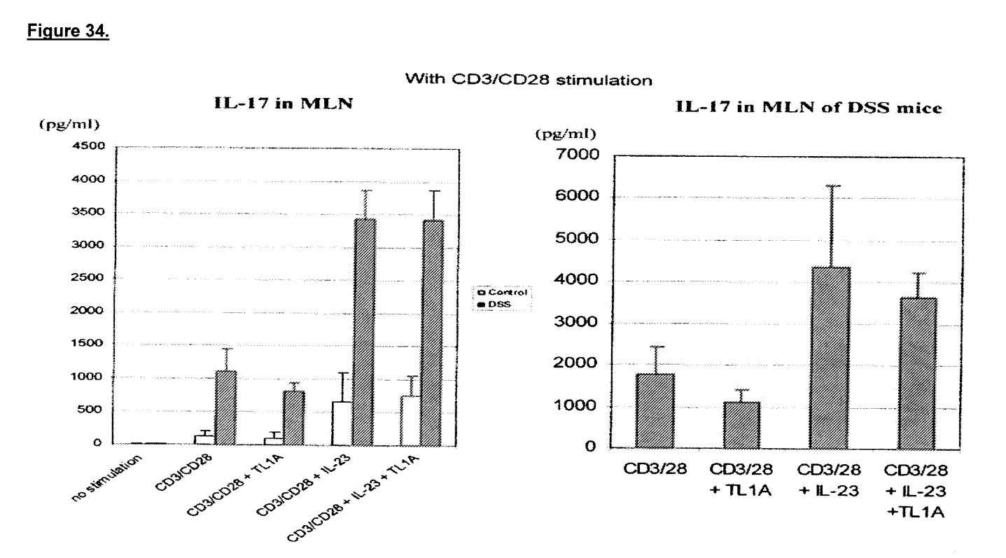

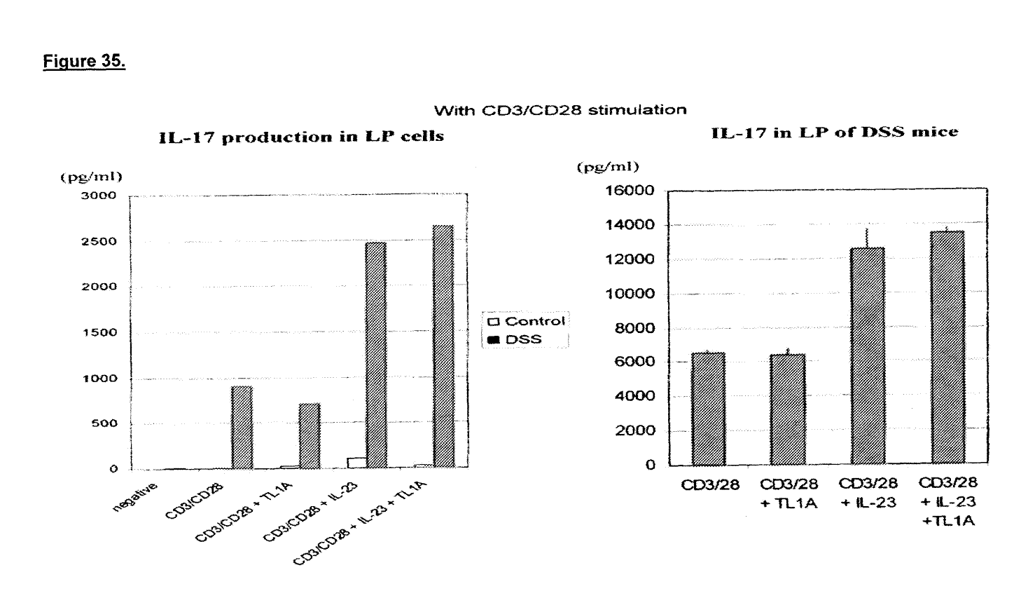

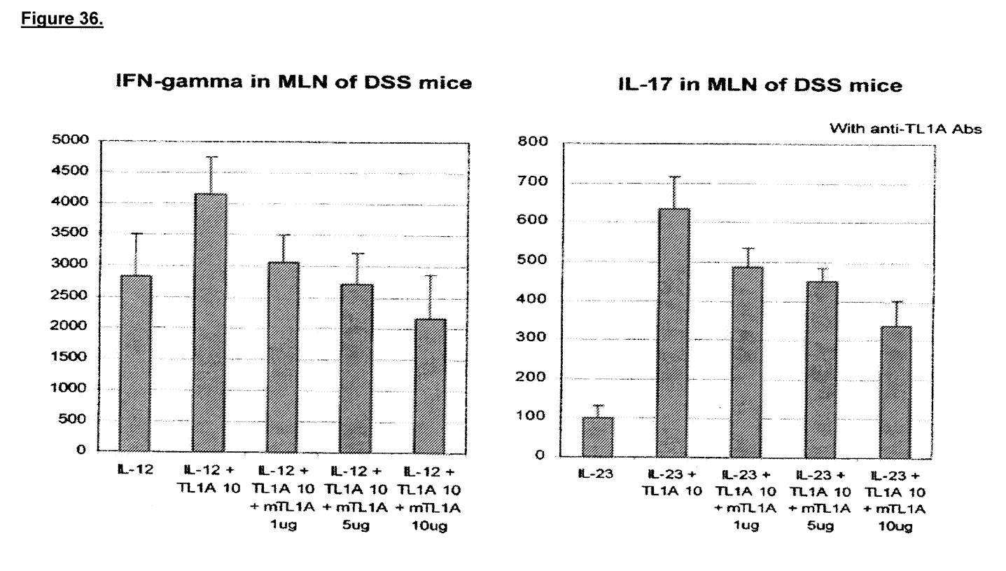

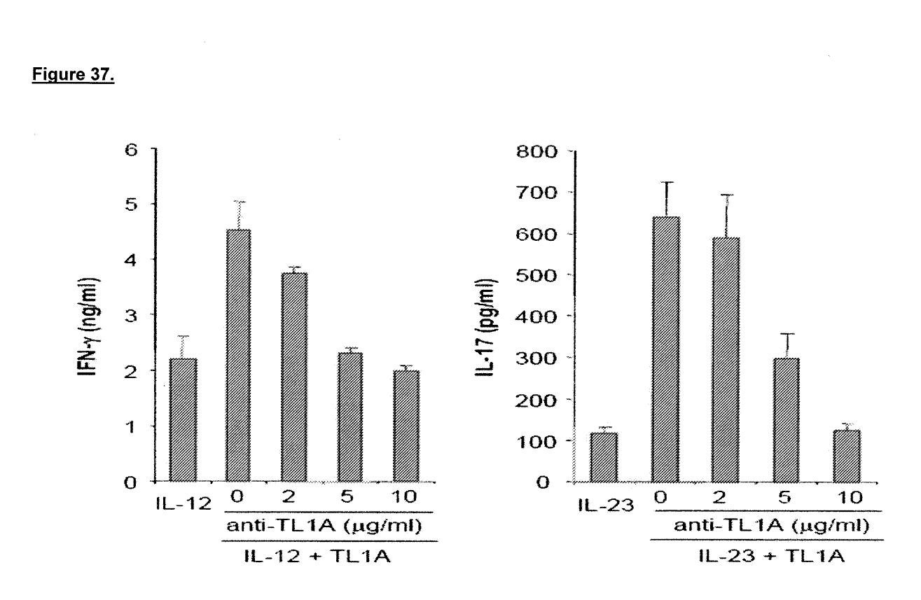

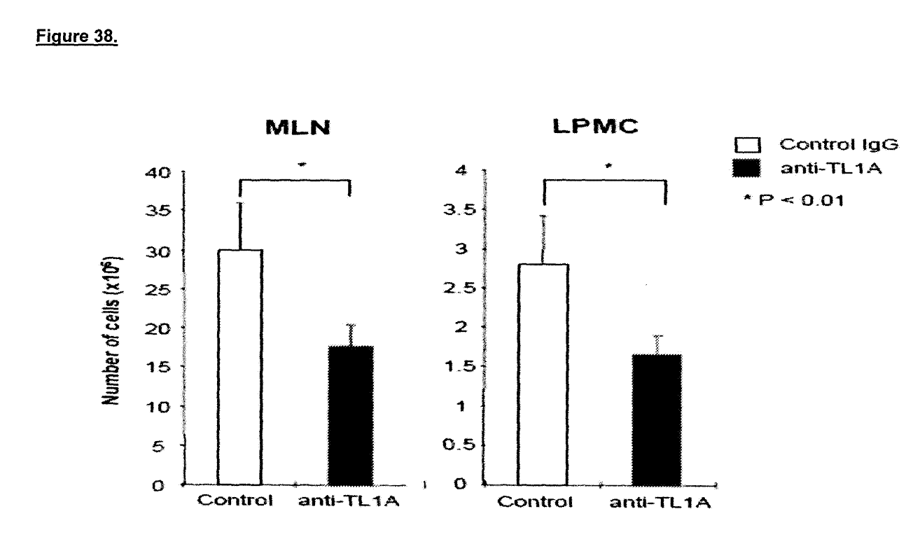

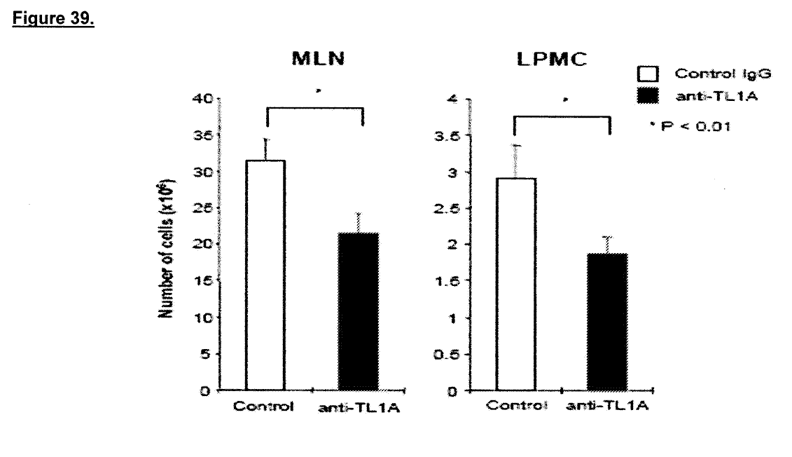

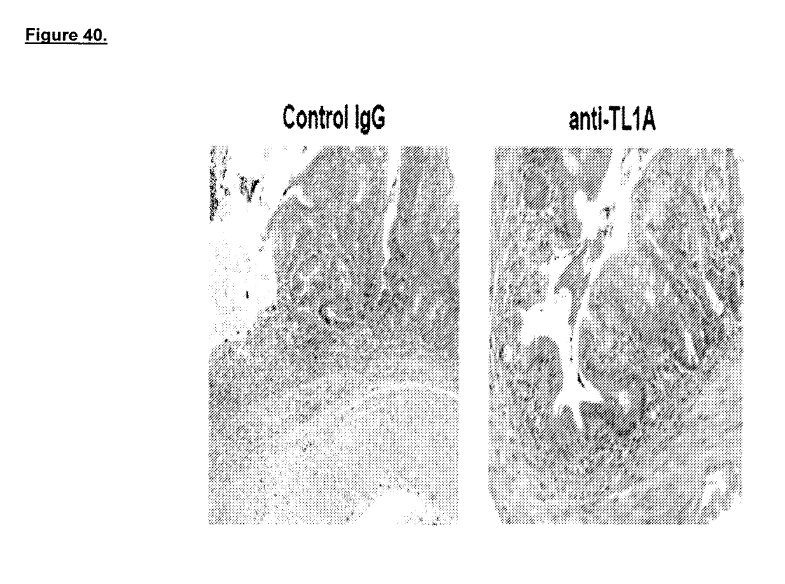

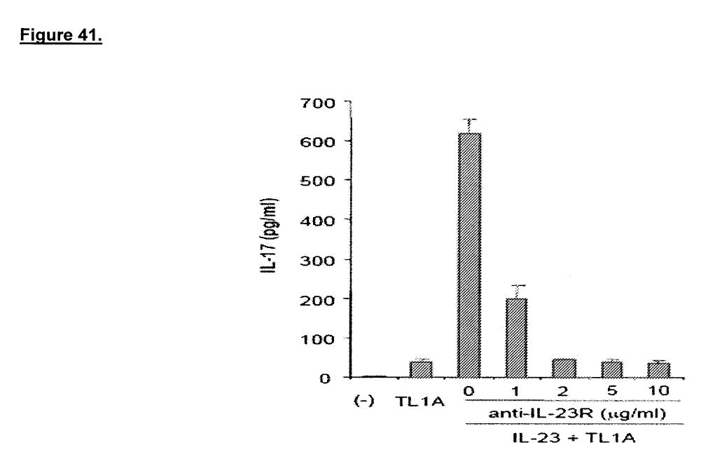

To determine whether induction of TL1A mRNA led to protein expression, TL1A was measured in supernatants following stimulation of monocytes and DC using the same agonists that were used in FIG. 4. Plate IC induced TL1A in supernatants beginning at 6 h. This increased rapidly thereafter with levels that closely paralleled peak mRNA levels. None of the TLR agonists, nor IFN-gamma, induced soluble TL1A. Since TLR signaling of both monocytes and DC induced inflammatory cytokines, the inventors examined whether the same agonists could induce IL-6 and/or TNF-α from monocytes and/or DC. The same supernatants from experiments were measured for IL-6 and TNF-α secretion. All ligands were capable of inducing high levels of IL-6 in monocytes and DC. Therefore, the lack of TL1A induction by these ligands was due to differences in pathways capable of inducing TL1A and IL-6 and not due to overall defective activation signaling. Furthermore, agonists of TLR4, TLR2 and TLR5 as well as Fc-gamma-R, all induced TNF-α secretion from both monocytes and DC. Therefore, TLR signaling could induce TNF-α, but not TL1A, where as Fc-gamma-R induced high levels of TL1A from both monocytes and DC.

### Example 10

### The T Cell Co-Stimulator TL 1A is Induced by Fc-Gamma-Receptor Signaling in Monocytes and Dendritic Cells: Fc-Gamma-R Signaling Induces the Expression of Membrane TL1A on Monocytes and Monocyte Derived Dendritic Cells

TNF secretion from monocytes is generated by membrane expression followed by TACE cleavage, which results in the release of TNF into the surrounding media. Using flow cytometry the inventors previously detected TL1A on the surface of T cells isolated from Crohn's disease lamina propria. By identifying the putative N-terminal trans-membrane domain in full length TL1A, it was demonstrated that similar to TNF-α, TL1A is expressed on the membrane. Since plate IC induced TL1A, mRNA and soluble protein, the inventors hypothesized that TL1A also might be expressed on the membrane of monocytes and DC stimulated by this pathway. Staining of monocytes stimulated for 16 h revealed that about half of the cells (range: 44.2 to 53.1% in four independent experiments) were strongly positive for TL1A, while DC stained at this time point showed an average of 12.6% positive cells (range: 8.6-17.4 in four individual experiments. To further characterize the TL1A surface expression upon IC stimulation the inventors performed time-course experiments. Stimulation of monocytes with IC revealed an increase of TL1A+ monocytes as early as 6 h after stimulation with IC (Table I). The number of TL1A+ monocytes was sustained for up to 16 h while the mean fluorescence intensity (MFI) increased over time. Taken together the increase in TL1A mRNA upon IC stimulation represents the increased numbers of TL1A+ monocytes and up-regulated TL1A surface expression as well as increased soluble TL1A.

TABLE 3
Time-course of TL1A induction on the cell surface of monocytes.
Monocytes were isolated from healthy volunteers. Cells were
incubated with IC for the indicated time points and stained for flow
cytometry with a TL1A specific Mab. A representative out of two
independent experiments is shown.

| | |
|---|---|
| control | IC stimulation |

| time point | % TL1A⁺ cells | MFI | % TL1A⁺ cells | MFI |
|---|---|---|---|---|
| 6 h | 2.7 | 938 | 28.5 | 968 |
| 12 h | 2.6 | 1036 | 21.9 | 1849 |
| 16 h | 1.77 | 797 | 32.2 | 1548 |

It was conceivable that TLR and/or IFN-gamma signaling could induce membrane-bound but not soluble TL1A. The inventors therefore investigated the surface expression of TL1A following TLR and IFN-gamma stimulation and found no induction of either surface or soluble TL1A by these pathways.

### Example 11

### The T Cell Co-Stimulator TL1A is Induced by Fc-Gamma-Receptor Signaling in Monocytes and Dendritic Cells: Fc-Gamma-R Signaling of Monocytes Enhances IFN-Gamma Production by CD4+ T Cells Through Induction of TL1A Expression

To determine whether Fc-gamma-R induced TL1A expression in monocytes has a functional consequence, the inventors used an IL-12/IL-18 primed CD4+ T cell culture system. To see whether monocyte induced TL1A could enhance IL-12/IL-18 primed CD4+ T cell production of IFN-gamma, CD4+ T cells from healthy donors were incubated overnight with IL-12/IL-18. The following day, T cells were co-cultured with autologous monocytes that had been pre-treated with or without IC. After an additional 48 h supernatants were collected and assayed for IFN-gamma. The inventors observed an at least 5-fold increase of IFN-gamma production by IL-12/IL-18 primed CD4+ T cells co-cultured with IC treated monocytes compared to untreated cells. To determine if this additional increase of IFN-gamma production was due to TL1A induced by Fc-gamma-R signaling, the inventors used blocking TL1A antibody in the co-cultures. In titration experiments the inventors determined the maximal inhibitory efficiency of this TL1A antibody to be approximately 50% under the same experimental conditions as used above. The additional increase of IFN-gamma production by CD4+ T cells co-cultured with IC treated monocytes was inhibited by an average of 36%. Taken together these data demonstrate that the TL1A induced by Fc-gamma-R signaling of monocytes is biologically active in vitro and results in enhanced IFN-gamma production by CD4+ T cells.

### Example 12

### The T Cell Co-Stimulator TL 1A is Induced by Fcgamma Receptor Signaling in Monocytes and Dendritic Cells: Cell Isolation and Culture

Blood was obtained from normal donors after informed consent in accordance with procedures established by the Cedars-Sinai Institutional Review Board. PBMC were isolated on standard Ficoll-Hypaque density gradients. Subsequent isolation of monocytes was performed using the Monocyte Isolation Kit II (Miltenyi Biotec, Auburn, Calif.) according to the manufacturer's protocol. Monocyte preparations were routinely >90% pure as determined by esterase stain (Sigma-Aldrich, St. Louis, Mo.). Monocytes were cultured in RPMI 1640 containing 2 mM glutamine and 25 mM HEPES buffer (Mediatech, Herndon, Va.), supplemented with 10% FBS, 50 μg/ml gentamicin (Omega Scientific, Tarzana, Calif.), and 0.25 μg/ml amphotericin B (Gemini Bioproducts, Woodland Hills, Calif.). To obtain monocyte-derived DC, GM-CSF (100 ng/ml) and IL-4 (30 ng/ml, both from Peprotech, Rocky Hill, N.J.) were added with 2-mercaptoethanol (50 μM), and the cells were cultured for 7 days (12). CD4+ T cells were isolated from PBMC using the human T lymphocyte Enrichment Set (BD Bioscience, San Diego, Calif.) and cultured for 24 h in the presence of human IL-12 (Peprotech, final concentration: 2 ng/ml) and human IL-18 (R&D Systems, final concentration: 50 ng/ml) in RPMI complete medium.

### Example 13

### The T Cell Co-Stimulator TL 1A is Induced by Fcgamma Receptor Signaling in Monocytes and Dendritic Cells: Stimulation of Dc and Monocytes

Monocytes and monocyte-derived DC were plated in 12-well plates and stimulated for 6, 12, 18 and 24 hrs. Monocyte and dendritic cell induction of mRNA and protein were accomplished using optimal activation concentrations of agonists defined either in the literature (IFNγ, Pam3CSK4) or by prior titration experiments (LPS, and CBir Flagellin) The concentrations used were IFNγ (R&D Systems, Minneapolis, Minn., 10 ng/ml), LPS (phenol-water extracted from E. coli K235, gift of Dr. S. N. Vogel, Department of Microbiology & Immunology, University of Maryland, Baltimore, Md., 50 ng/ml), Pam3CSK4 (Invitrogen, San Diego, Calif., 300 ng/ml) and full-length recombinant CBir1 Flagellin (10 μg/ml) respectively. Plate-bound, cross-linked human IgG (plate IC) was prepared by incubating 1 ml of human IgG (Jackson ImmunoResearch, West Grove, Pa.) in PBS (0.5 mg/ml) per well of a 12-well plate for 1 h to overnight, followed by washing with PBS, and incubation with 750 μl mouse anti-human IgG (Jackson) in PBS (20 μg/ml) for 1 h. Coated plates were washed with PBS before plating cells for stimulation.

### Example 14

### The T Cell Co-Stimulator TL1A is Induced by Fcgamma Receptor Signaling in Monocytes and Dendritic Cells: Co-Culture of CD4+ T Cells with Monocytes

After 24 h culture of CD4+ T cells medium was replaced with fresh medium supplemented with IL-12 and IL-18 and cells were added to autologous monocytes. Prior to co-cultures, monocytes were incubated with immune complex (IC) or left untreated overnight (18 h). At the time of coculture, recombinant human TL1A (final concentration: 50 ng/ml), TL1A antibodies (final concentration: 15 μg/ml) or control antibodies (IgG2b, eBioscience, final concentration: 15 μg/ml) were added to the cells. Co-cultures were incubated for 2 days and supernatants were harvested and analyzed for IFN-gamma production by ELISA.

### Example 15

### The T Cell Co-Stimulator TL1A is Induced by Fcgamma Receptor Signaling in Monocytes and Dendritic Cells: Real-Time PCR Analyses

Total RNA was isolated from monocytes and monocyte-derived DC using Trizol (Invitrogen Life Technologies, Carlsbad, Calif.) according to the manufacturer's protocol. TL1A and β-Actin transcripts were amplified by quantitative real-time RT-PCR with TaqMan probes and primers designed using Beacon Design 4.0 (Premier Biosoft International, Palo Alto, Calif.). TL1A (TNFSF15) is the predominant product of the 4 exon tl1a TNF superfamily gene, although another transcript (encoding VEGI) can be produced from a transcript initiated at the 3′ end of the gene (1), and thus, chose a primer/probe set specific for TL1A: the forward primer (in exon 1) was CTTCCTTGCAGGACTCACCAC (SEQ. ID. NO.: 12), the reverse primer (in exon 2) was GCTGATGTGAAGGTGCAAACTC (SEQ. ID. NO.: 13, and the probe (in exon 1) was 5′-3′ ACCTGCTTGTCAGCCAGCTCCGG (SEQ. ID. NO.: 14). The β-Actin forward primer was GACTACCTCATGAAGATCCTCACC (SEQ. ID. NO.: 15), the reverse primer was TCTCCTTAATGTCACGCACGATT (SEQ. ID. NO.: 16), and the probe was 5′-3′ CGGCTACAGCTTCACCACCACGGC (SEQ. ID. NO.: 17). 500 ng of total RNA was used in each RT reaction, with oligo-dT as primer, using the Omniscript kit and protocol (Qiagen Inc, Valencia, Calif.). PCR was done on ¼ the RT reaction in duplicate as follows: 50° C. for 2 min, 95° C. for 2 min, then 50 cycles at 95° C. for 15s, and 60° C. for 1 min. Assays were performed following the pre-developed TaqMan assay reagents protocol for Platinum qPCR mix (Invitrogen Life Technologies, Carlsbad, Calif.) in an iCycler (Bio-Rad, Hercules, Calif.). The iCycler Optical system Interface (Bio-Rad) was used to analyze samples. Duplicates differing by less than one cycle were averaged and amount of transcript was analyzed as 2E (CT Beta-actin-CT TL1A) for each sample and expressed as % of Beta-actin. Statistical significance was determined by Student's t test. P<0.01 was considered to be statistically significant.

### Example 16

### The T Cell Co-Stimulator TL 1A is Induced by Fcgamma Receptor Signaling in Monocytes and Dendritic Cells: ELISA

TL1A was quantified in undiluted supernatants from stimulated cells using an ELISA and Monoclonal antibodies. The capture monoclonal antibody was MAb 04H08, the detector was biotinylated MAb 16H02, and the standard was recombinant soluble TL1A (aa72-251). Biotinylated detector Ab was bound by streptavidin-HRP and plates were developed by a standard amplified color reaction and read in a plate reader (Molecular Devices, Sunnyvale, Calif.). This ELISA has a detection limit of 0.2 ng/ml TL1A. Human IL-6 or TNF-α concentrations were measured by ELISA (eBioscience, San Diego, Calif.). Supernatants from cells treated with various stimuli were harvested after 6 h (TNF-α) or 18 h (IL-6), respectively. Human INF-gamma was measured by ELISA.

### Example 17

### The T Cell Co-Stimulator TL1A is Induced by Fcgamma Receptor Signaling in Monocytes and Dendritic Cells: Flow Cytometry

Monocytes or dendritic cells stimulated by plate-bound IC for 6-16 h were stained, washed, stained with biotinylated goat antimouse IgG2b (CALTAG, Burlingame, Calif.), washed and stained with APC conjugated streptavidin (CALTAG). Fixed cells were analyzed on a Cyan (Dako-Cytomation, Fort Collins, Colo.) flow cytometer.

### Example 18

### Use of the TL 1A Genetic Haplotype and Serologic Expression for Targeting Anti-TL1A Therapy in Patients with Inflammatory Bowel Disease

The inventors discovered a new technique for assessing the level of TL1A expression in normal subjects and patients with inflammatory bowel disease, and the significance of this level as an indicator of a specific form of inflammation. This invention is believed to have relevance for diseases other than IBD, in which TL1A may play a role in inflammation, such as rheumatoid arthritis, multiple sclerosis, psoriasis and/or any other chronic inflammatory disease. TL1A (TNF super family member 15) is a recently described TNF-like molecule that is expressed in myeloid cells and T-cells. The inventors have shown that there is markedly increased expression of this molecule in the mucosa of Crohn's disease patients and some patients with ulcerative colitis. In addition, expression of this molecule has been shown to be similarly increased in the synovium of patients with rheumatoid arthritis and in lesions of patients with psoriasis. The inventors recently defined haplotypes of the TL1A gene and have demonstrated an association with one of these haplotypes, haplotype16 (which appears to be dominant). When monocytes from patients expressing this haplotype are signaled via the Fc-gamma-receptor, TL1A production is high. Fc-gamma-receptor stimulated monocytes from patients expressing this haplotypes have augmented T cell production of IFN-gamma. Patients with the haplotype 1, i.e., 1-1 or non-16, have very low levels of TL1A and stimulated monocytes do not augment T cell production of IFN-gamma. Another aspect of this invention is the inventors' discovery that antibodies to TL1A can attenuate chronic colitis in the DSS mouse model.

Thus, the inventors have found that TL1A is an important contributor to chronic inflammation in a subset of patients with Crohn's disease who express TL1A haplotype 16 and who also express the serologic marker OmpC. Anti-TNF therapies and therapeutics designed to inhibit TL1A would be targeted to patients who have this particular haplotype and/or OmpC positive serologies. In contrast, patients who have the haplotype 1 or 1 non-16, who express low levels of TL1A are unlikely to benefit from this approach. The invention represents an approach for blockage of this molecule in a patient-specific fashion.

### Example 19

### TNFSF15 Regulation of Th1 and Th17 Function

TL1A is a recently identified tumor necrosis factor-like molecule and its receptor death receptor (DR) 3 expression is increased in the mucosa of Crohn's disease (CD) patients. To determine the possible role of TL1A in CD, the inventors investigated its role in a mouse model of chronic colitis. TL1A, DR3, IFN-gamma and IL-17 were significantly increased in chronic colitis. TL1A up-regulated both IFN-gamma and IL-17 production from CD4⁺ T cells in the gut associated lymphoid tissue (GALT) of diseased mice. Furthermore, both IFN-gamma and IL-17 production in the GALT induced by IL-12 and IL-23 respectively, was synergistically enhanced by TL1A. Neutralizing anti-mouse TL1A antibodies significantly attenuated chronic colitis by down-regulation of both T-helper (T_{H}) 1 and T_{H}17 activation. These results show that TL1A is an important modulator of the development of chronic mucosal inflammation. The central role of TL1A represents an attractive, novel therapeutic target for the treatment of CD patients.

### Example 20

### TNFSF15 Regulation of Th1 and Th17 Function: Characterization of DSS Induced Chronic Colitis

Since TL1A has been shown to be important for mucosal T_{H}1 response, the inventors used the murine model of DSS-induced chronic colitis to test the role of TL1A in mucosal inflammation. This T_{H}1 mediated chronic colitis was induced by administration of four cycles of 3% DSS drinking water on days 1-5, 8-12, 15-19, and 22-26 to C57BL/6 mice. Loss of body weight began at day 5 and was caused by acute colitis. Maximum weight loss was seen at day 12 but mice regained body weight after 2 cycles of DSS. However, diarrhea was observed throughout all four cycles of DSS-administration. Mice were sacrificed at day 29 and the colon length was measured from cecum to rectum. Colon length has been described as a good indicator for severity of colitis and cecum and colon of DSS-treated mice showed signs of severe colitis including significantly shortened colon length. Mononuclear cell numbers from mesenteric lymph node (MLN) and lamina propria (LP) were increased due to infiltration of inflammatory cells in DSS-treated mice. On histological examination, crypt damage, colonic epithelial cell hyperplasia, crypt elongation and infiltration of inflammatory cells were observed in cecum and colon of DSS-treated mice. Cellular composition of MLN and LP mononuclear cells (LPMC) were analyzed by flow cytometry. In MLN and LP, B cells are significantly expanded. The percentage of CD4⁺ and CD8⁺T cells are relatively decreased in MLN while the absolute numbers of CD4⁺T cells are increased in DSS-treated mice (control vs. DSS: 2.8×10⁶ vs. 6.2×10⁶, p<0.01). In LP, CD4⁺T cell, especially CD4⁺CD45RB^{low} memory T cells (control vs. DSS: 11.3% vs. 16.3%), were significantly increased in DSS-treated mice. This result suggested memory T cells were infiltrated in LP and involved in development of chronic colitis.

### Example 21

### TNFSF15 Regulation of Th1 and Th17 Function: T_{H}1 and T_{H}17 Cytokine Profile of DSS-Induced Chronic Colitis

IFN-gamma is mainly produced by CD4⁺T cells (T_{H}1 cells), and IL-17 is produced by CD4⁺T cells (T_{H}17 cells) that are distinct from the classical T_{H}1 and T_{H}2 lineage. Both IFN-gamma and IL-17 are key mediators in several autoimmune diseases such as rheumatoid arthritis, experimental autoimmune encephalomyelitis, and IBD. Therefore, the inventors examined IFN-gamma and IL-17 production of T cells in the GALT of DSS induced chronic colitis. Involvement of cytokines was examined by evaluating the expressions of several T_{H}1 and T_{H}17 polarizing cytokines in MLN and colonic mucosa by Real-Time-PCR. The inventors found IFN-gamma, IL-17, and TNF-α mRNA expressions to be significantly increased in DSS-treated mice compared to untreated controls. In addition, high expression of these cytokines was associated with the severity of colitis. To assess the overall potential of cytokine production by T cells in GALT, the inventors examined the production of IFN-gamma and IL-17 in MLN and LPMC after stimulation with anti-CD3ε and anti-CD28 Abs. The inventors found that anti-CD3ε and anti-CD28 Abs mediated IFN-gamma and IL-17 production was increased in DSS-treated mice at the protein level.

### Example 22

### TNFSF15 Regulation of Th1 and Th17 Function: Increased TL1A and DR3 Expression in DSS-Induced Chronic Colitis

To evaluate TL1A and DR3 expression in DSS colitis, the inventors performed Real-Time PCR in MLN and colons of DSS-treated and untreated mice. TL1A mRNA was significantly increased in both MLN and colon of DSS-treated compared with untreated mice. DR3 expression, which has been reported to be mainly expressed by CD4⁺CD45RB^{low} memory T cells, was also increased in DSS colitis. FACS analysis revealed that CD11c^{high} MHC class II⁺DCs expressed TL1A in MLN from DSS-treated mice. These results indicate that an increase of mucosal TL1A and DR3 expression is associated with DSS-induced chronic inflammation as was shown in human CD.

Because the interaction of APC derived TL1A and DR3 expressed on T cells was shown to induce T cell activation in mucosa, the inventors investigated the effect of TL1A on MLN and LPMC. Mononuclear cells were isolated from MLN and LP in DSS-treated and untreated mice and cultured with 10 ng/ml TL1A for 72 h. Although the inventors did not find any effect of TL1A in untreated mice, IFN-gamma and IL-17 were produced from MLN and LP cells from DSS-treated mice and the production was greatly enhanced by stimulation with TL1A. TL1A enhanced much more cytokine production from LPMC compared with MLN cells, possibly because of the increased number of memory T cells in LP of DSS-treated mice. Therefore, elevated levels of TL1A and DR3 expression in DSS-treated mice can be involved directly in enhanced mucosal expression of IFN-gamma (T_{H}1) and IL-17 (T_{H}17).

### Example 23

### TNFSF15 Regulation of Th1 and Th17 Function: TL1A Synergistically Enhances IL-12-Induced IFN-Gamma and IL-23-Induced IL-17 Secretion from Mucosal CD4⁺T Cells

IL-12 induces the differentiation of naïve CD4+ T cell into IFN-gamma producing T_{H}1 cells through activation of STAT4. In contrast, IL-23 is responsible for the differentiation and expansion of IL-17 producing T_{H}17 cells. Previously it was shown that TL1A enhances the production of IFN-gamma from CD4⁺T cells stimulated with IL-12 alone or in combination with IL-18 in human PBMC and murine splenocytes. To investigate the role that TL1A might play in both IFN-gamma and IL-17 production in the mucosa of DSS-treated mice, the inventors examined the synergistic effect of TL1A with IL-12 or IL-23 in MLN and LP on the production of IFN-gamma and IL-17. Mononuclear cells were isolated from MLN and LP from untreated and DSS-treated mice and stimulated with TL1A, IL-12, IL-23 alone, TL1A plus IL-12, or TL1A plus IL-23. Stimulation with IL-12 plus TL1A enhanced IFN-gamma production in both untreated and DSS-treated mice. Interestingly, the inventors found that stimulation with IL-23 plus TL1A could significantly up-regulate IL-17 production compared to stimulation with IL-23 alone. The enhancing effects of TL1A on the production of IFN-gamma or IL-17 were much more pronounced in DSS-treated mice compared to untreated mice and in particular in the LP of DSS-treated mice.

To confirm the effect of TL1A on CD4^{÷}T cell, the inventors performed intracellular staining of LPMC under conditions driving either T_{H}1 or T_{H}17 differentiation. Cells isolated from LP of DSS-treated mice were cultured with TL1A, IL-12, IL-23 alone, TL1A plus IL-12, or TL1A plus IL-23 for 72 h. CD4⁺IFN-gamma-producing (T_{H}1) cells and IL-17-producing (T_{H}17) cells were identified after PMA re-stimulation. TL1A alone increased both the numbers of IFN-gamma producing cells and IL-17-producing cells (IFN-gamma: 0.67 vs. 1.00, IL-17: 0.47 vs. 0.89, p<0.03). This result revealed that TL1A could up-regulate IFN-gamma and IL-17 production in both T_{H}1 and T_{H}17 via DR3 pathway even in the absence of cytokines known to drive T_{H}1 and T_{H}17 differentiation. Moreover, IL-12 plus TL1A, and IL-23 plus TL1A further increased numbers of IFN-gamma producing cells and IL-17-producing cells, respectively (IFN-gamma: 4.39 vs. 8.34, IL-17: 1.53 vs. 2.40). These data were consistent with the data on soluble cytokine production from MLN and LPMC. The inventors obtained the same results in MLN, although the total numbers of IFN-gamma-producing cells and IL-17-producing cells were lower. Interestingly, the inventors observed a population of IFN-gamma and IL-17 double positive cells when LPMC were stimulated with IL-23 alone or IL-23 plus TL1A, but not when stimulated with IL-12. To further investigate this finding, the inventors examined the effect of IL-23 and IL-23 plus TL1A on IFN-gamma production, and the effect of IL-12 and IL-12 plus TL1A on IL-17 production. The data showed that IL-23 and IL-23 plus TL1A could up-regulate IFN-gamma production, however, IL-12 and IL-12 plus TL1A inhibited IL-17 production. These results were consistent with the inventors' data of intracellular staining. These findings show that IL-23 and TL1A can induce both T_{H}1 and T_{H}17 differentiation, and TL1A may enhance both IFN-gamma and IL-17 production from T_{H}17 cells, but not from T_{H}1 cells.

### Example 24

### TNFSF15 Regulation of Th1 and Th17 Function: Increase of IL-6 Production from Mucosal CD4⁺T Cell by the Synergistic Effect of TL1A with IL-23

Previous reports have shown that IL-6 plays an important role in the pathogenesis of chronic IBD, with a recent report showing that IL-23 enhanced IL-6 production from T_{H}17 cells. Furthermore, IL-6 has been demonstrated to play a crucial role in the differentiation of T_{H}17 cells from naïve T cells. To investigate IL-6 production in DSS-treated and untreated mice, MLN and LPMC were cultured with anti-CD3E and anti-CD28 Abs for 72 h. IL-6 production from T cells was significantly increased in DSS-treated mice. To assess whether TL1A enhance IL-6 production, the inventors measured IL-6 production from MLN and LPMC, which were cultured with or without TL1A. In this data, the inventors also found TL1A up-regulated IL-6 production. Furthermore, to investigate the synergetic effect of TL1A to IL-6, mononuclear cells from MLN and LPMC were cultured with IL-12, IL-23 alone, TL1A plus IL-12, or TL1A plus IL-23. IL-23, but not IL-12 up-regulated IL-6 production. The data showed IL-6 were produced by T cells and TL1A significantly enhanced IL-23-induced IL-6 production as well as IL-17. However, TL1A did not enhance TNF-α production.

### Example 25

### TNFSF15 Regulation of Th1 and Th17 Function: Attenuation of the Development of DSS-Induced Chronic Colitis by the Neutralization of TL1A

To investigate the role of TL1A in this T_{H}1/T_{H}17 mediated chronic colitis, the inventors utilized anti-mouse TL1A monoclonal antibodies (mAbs) in the mouse model of chronic colitis. The neutralizing effect of anti-TL1A mAbs was assessed in vitro using MLN and LPMC and co-culturing cells with IL-12 or IL-23 plus 10 ng/ml TL1A. Increasing concentration of neutralizing anti-TL1A mAbs was added to the cultures. 10 μg/ml anti-TL1A mAbs completely neutralized the enhancing effect of TL1A on IFN-gamma and IL-17 production. Since the data showed that TL1A enhanced mucosal T cell activation and IFN-gamma, IL-17, and IL-6 cytokine production in chronic DSS colitis, the inventors hypothesized that TL1A was centrally involved in development of chronic colitis. To examine this hypothesis, neutralizing anti-TL1A m Abs or control IgG were administered once a week in DSS-treated mice starting at day 1. Chronic colitis was induced by administration of 3% DSS drinking water on days 1-5, 8-12, 15-19, and 22-26. 500 μg of mAbs were administered by repeated intraperitoneal injection on days 1, 8, 15, and 22. Mice were sacrificed at day 29 and colitis was evaluated in a blinded fashion. Administration of anti-TL1A mAbs lead to a significant protection against DSS induced colitis, as indicated by significant attenuation of weight loss. Furthermore, upon macroscopic examination mice treated with control IgG displayed a significant shortening of the cecum and colon length compared to the anti-TL1A mAbs treatment group. On histological examination, the numbers of infiltrating cells, degree of mucosal injury, and edema were reduced in the anti-TL1A mAbs treatment group. The histological scores of the cecum and colon were significantly lower in anti-TL1A mAbs treatment group than in control IgG group.

To determine the effect of anti-TL1A mAbs treatment on cell number and cytokine production, mononuclear cells were isolated from MLN and LP in mice from anti-TL1A mAbs treatment group and control group. Anti-TL1A mAbs treatment group showed a significant reduction in the numbers of mononuclear cell in MLN and LP compared to the control IgG treatment group. The effect of anti-TL1A mAbs was also associated with a decrease of B cell and memory CD4⁺T cell numbers. IFN-gamma, IL-17, and IL-6 productions produced by anti-CD3ε and anti-CD28 stimulated T cells isolated from MLN and LP of DSS-treated mice, were significantly decreased by the in vivo administration of anti-TL1A mAbs compared to control IgG. Thus, in addition to a significant modulation of chronic colitis, anti-TL1A mAbs suppressed the production of IFN-gamma (T_{H}1) and IL-17/IL-6 (T_{H}17) cytokines from mucosal T cells.

### Example 26

### TNFSF15 Regulation of Th1 and Th17 Function: Conclusions

The inventors demonstrate the role of TL1A in the development of colitis. Interestingly, TL1A strongly enhanced both IFN-gamma and IL-17/IL-6 production from mucosal CD4⁺T cells induced by IL-12 and IL-23 respectively. Neutralization of TL1A inhibited the infiltration of mucosal T cells and production of IFN-gamma, IL-17, and IL-6 and attenuated chronic inflammation. These results show that TL1A is a central immune modulator for activation of mucosal CD4⁺T cells with T_{H}1/T_{H}17 response in the development of colitis.

1. A method of diagnosing susceptibility to a subtype of Crohn's Disease in an individual, comprising:
determining the presence or absence of one or more risk variants at the TNFSF15 locus in the individual,
wherein the presence of one or more risk variants at the TNFSF15 locus is diagnostic of susceptibility to the subtype of Crohn's Disease.

2. The method of claim 1, wherein said individual is a child.

3. The method of claim 1, wherein the subtype is associated with the absence of NOD2 risk variants.

4. The method of claim 1, wherein the subtype further comprises complicated small bowel disease phenotype.

5. The method of claim 1, wherein the subtype further comprises internal penetrating and/or stricturing disease phenotype.

6. The method of claim 1, wherein one of said one or more risk haplotypes at the TNFSF15 locus in the individual is haplotype A.

7. The method of claim 1, wherein the one or more risk haplotypes at the TNFSF15 locus in the individual comprises one or more variant alleles selected from SEQ. ID. NO.: 3, SEQ. ID. NO.: 4, SEQ. ID. NO.: 5, SEQ. ID. NO.: 6, and SEQ. ID. NO.: 7.

8. A method of determining in an individual a low probability relative to a healthy individual of developing inflammatory bowel disease, comprising:
determining the presence or absence of one or more protective haplotypes at the TNFSF15 locus,
wherein the presence of one or more protective haplotypes at the TNFSF15 locus is diagnostic of the low probability relative to the healthy individual of developing inflammatory bowel disease.

9. The method of claim 8, wherein the individual is a child.

10. The method of claim 8, wherein the individual is non-Jewish.

11. The method of claim 8, wherein the inflammatory bowel disease further comprises complicated small bowel disease phenotype.

12. The method of claim 8, wherein the inflammatory bowel disease further comprises internal penetrating and/or stricturing disease phenotype.

13. The method of claim 8, wherein the inflammatory bowel disease further comprises Crohn's Disease.

14. The method of claim 8, wherein the inflammatory bowel disease further comprises ulcerative colitis.

15. The method of claim 8, wherein one of said one or more protective haplotypes at the TNFSF15 locus is haplotype B.

16. The method of claim 8, wherein the one or more protective haplotypes at the TNFSF15 locus comprise one or more variant alleles selected from SEQ. ID. NO.: 3, SEQ. ID. NO.: 4, SEQ. ID. NO.: 5, SEQ. ID. NO.: 6, and SEQ. ID. NO.: 7.

17-35. (canceled)


SEQUENCE LISTING (17 sequences)

SEQ ID NO: 1 - DNA, 2011 bp, Homo sapiens
ggaaaagggaaggaggagactgagtgattaagtcacccactgtgagagctggtcttctatttaatgggggctctctctgcccaggagtcagaggtgcctccaggagcagcaggagcatggccgaggatctgggactgagctttggggaaacagccagtgtggaaatgctgccagagcacggcagctgcaggcccaaggccaggagcagcagcgcacgctgggctctcacctgctgcctggtgttgctccccttccttgcaggactcaccacatacctgcttgtcagccagctccgggcccagggagaggcctgtgtgcagttccaggctctaaaaggacaggagtttgcaccttcacatcagcaagtttatgcacctcttagagcagacggagataagccaagggcacacctgacagttgtgagacaaactcccacacagcactttaaaaatcagttcccagctctgcactgggaacatgaactaggcctggccttcaccaagaaccgaatgaactataccaacaaattcctgctgatcccagagtcgggagactacttcatttactcccaggtcacattccgtgggatgacctctgagtgcagtgaaatcagacaagcaggccgaccaaacaagccagactccatcactgtggtcatcaccaaggtaacagacagctaccctgagccaacccagctcctcatggggaccaagtctgtatgcgaagtaggtagcaactggttccagcccatctacctcggagccatgttctccttgcaagaaggggacaagctaatggtgaacgtcagtgacatctctttggtggattacacaaaagaagataaaaccttctttggagccttcttactataggaggagagcaaatatcattatatgaaagtcctctgccaccgagttcctaattttctttgttcaaatgtaattataaccaggggttttcttggggccgggagtagggggcattccacagggacaacggtttagctatgaaatttggggcccaaaatttcacacttcatgtgccttactgatgagagtactaactggaaaaaggctgaagagagcaaatatattattaagatgggttggaggattggcgagtttctaaatattaagacactgatcactaaatgaatggatgatctactcgggtcaggattgaaagagaaatatttcaacaccttcctgctatacaatggtcaccagtggtccagttattgttcaatttgatcataaatttgcttcaattcaggagctttgaaggaagtccaaggaaagctctagaaaacagtataaactttcagaggcaaaatccttcaccaatttttccacatactttcatgccttgcctaaaaaaaatgaaaagagagttggtatgtctcatgaatgttcacacagaaggagttggttttcatgtcatctacagcatatgagaaaagctacctttcttttgattatgtacacagatatctaaataaggaagtatgagtttcacatgtatatcaaaaatacaacagttgcttgtattcagtagagttttcttgcccacctattttgtgctgggttctaccttaacccagaagacactatgaaaaacaagacagactccactcaaaatttatatgaacaccactagatacttcctgatcaaacatcagtcaacatactctaaagaataactccaagtcttggccaggcgcagtggctcacacctgtaatcccaacactttgggaggccaaggtgggtggatcatctaaggccgggagttcaagaccagcctgaccaacgtggagaaaccccatctctactaaaaatacaaaattagccgggcgtggtagcgcatggctgtaatcctggctactcaggaggccgaggcagaagaattgcttgaactggggaggcagaggttgcggtgagcccagatcgcgccattgcactccagcctgggtaacaagagcaaaactctgtccaaaaaaaaaaaaaaaaaaaa

SEQ ID NO: 2 - protein, 251 aa, Homo sapiens
MAEDLGLSFGETASVEMLPEHGSCRPKARSSSARWALTCCLVLLPFLAGLTTYLLVSQLRAQGEACVQFQALKGQEFAPSHQQVYAPLRADGDKPRAHLTVVRQTPTQHFKNQFPALHWEHELGLAFTKNRMNYTNKFLLIPESGDYFIYSQVTFRGMTSECSEIRQAGRPNKPDSITVVITKVTDSYPEPTQLLMGTKSVCEVGSNWFQPIYLGAMFSLQEGDKLMVNVSDISLVDYTKEDKTFFGAFLL

SEQ ID NO: 3 - DNA, 601 bp, Homo sapiens
ccccaaatttcatagctaaaccgttgtccctgtggaatgccccctactcccggccccaagaaaacccctggttataattacatttgaacaaagaaaattaggaactcggtggcagaggactttcatataatgatatttgctctcctcctatagtaagaaggctccaaagaaggttttatcttcttttgtgtaatccaccaaagagatgtcactgacgttcaccattagcttgtccccttcttgcaaggagaacatggctccgaggtagatgggctggaaccagttgctacctacttcgcayacagacttggtccccatgaggagctgggttggctcagggtagctgtctgttaccttggtgatgaccacagtgatggagtctggcttgtttggtcggcctgcttgtctgatttcactgcactcagaggtcatcccacggaatgtgacctgggagtaaatgaagtagtctcccgactctgggatcagcaggaatttgttggtatagttcattcggttcttggtgaaggccaggcctagttcatgttcccagtgcagagctgggaactgatttttaaagtgctgtgtgggagtttgtctcaca

SEQ ID NO: 4 - DNA, 601 bp, Homo sapiens
tcttctgcaaaaccaagggctgtcatttctattttgcaattttctcatggtgtttcaattccaggccttggcatgctgctctcctggattctttcatcagcccctggcagggtctttgtacttcagttttctccttttccttgatctatttcctggcctgccttcagagggctttttctaaagcatcactttatctagatactacactgcttataaaccttcaatggctcctcatggctcttaagacccagtccaatctcattttgtcttggcattcaaagtcctaacttatcccagtctygctatccattatttacttctctctaagccctctgtgttcccagccatgagaggcaaccactgtgaattgattcaaggtatctgaagaaacagagttaagtgtagttactttattgaattagtagggacacagagacctggcccagaaatgggagaatcagaagaaattgctcctgaacagagtcttagaggtcctgcaggtactagccaggtgataaaagaggtaatgactattccagacaaggccaacagcttgcacaacagcaaatacctcaataccctgcatctgtgatgtggtgtc

SEQ ID NO: 5 - DNA, 601 bp, Homo sapiens
cctacaacagaaaccaagtttggtttgaggaaaaaaaaaaaaaaaaaaaaaagaagaagaaggagaagaagaaagctttcccaagcatatttatatacagtatgctcatgtgctccttccttcgtttacagaaggaagttaggaaagtccctgaaggaggagagaaagaattcatcaagtcagtgggtggggcaaattaaaatatacctgttccctgcactggaggcttaccagctgtgccagtctggggagtgtgcttctggaagtgaaagtgagggatgagaggtgtgtggtttgcagrttgggaaacggaaatcacatttgcatcagctctttgcaaagtgctgcctagccctctgtcattttgaacctcatagaaattcattctcagtgtacagatgggaatagcaaagttctaaaaggtgaaggcacttgtcctaggtcatccaaggatgaagacagaggagctaggaagatgacctagttctaaatcacggcttggagttgtaacctctagcacatgactgcccatgaaaggaaagtatttccagtctgcattgaccattgtttaatcagagtatgaggccacagatcgaggtga

SEQ ID NO: 6 - DNA, 682 bp, Homo sapiens
atgagaggtgtgtggtttgcagattgggaaacggaaatcacatttgcatcagctctttgcaaagtgctgcctagccctctgtcattttgaacctcatagaaattcattctcagtgtacagatgggaatagcaaagttctaaaaggtgaaggcacttgtcctaggtcatccaaggatgaagacagaggagctaggaagatgacctagttctaaatcacggcttggagttgtaacctctagcacatgactgcccatgaaaggaaagtatttccagtctgcattgaccattgtttaatcagagtaygaggccacagatcgaggtgactgtctgtgagggtagaacattaaccactactccctgattagtctaaagttaattgatcatgtgatgtgctttgcctgcagttgggtgtgggggccacaacatgtaataaaagattatatttattaagtgcttactttgtgccaatcactgctctaagttaaatacatcaataaaattattcaatcctgagataaattttgtgacaataaagctatcattctcattttacacataagaaaataaagcacagacggcaagtggtagagccaggattgggactcaggcaggctggctgctggcttcttcaccatcacacttcaccataggtactgctggatcatgcatgttcataaatacc

SEQ ID NO: 7 - DNA, 601 bp, Homo sapiens
aatgtctttgtatcagaaggaaactgtctgaaggtcagccccctctccactcctgaaaaggcataattcagagttggcatttttagttttgcttttctatttccaaatatgaaacagacaaggagtcaggagacccatcacgaacttgctatgtgacttcaggctgttcactcccctgtctctggtctccaggaaaacaagagggctgagaatccacccagatccatggtttttgtgtatctggggagtgggctattccattgaaatgtgtgttttgatgatcatggctaagtgggacttyagtgactcaaaccctgtgttcagatgaagcctgctcagatttctcctataagcgtagaagaaatgagggttttggaggcccaggctggggtctcactggacagtcttgagaggtgggagtgaatgtgaactctggagcacattggtttccacgtgtccctctgttgtgtaaccccaggcaaatcatggagcctccttaggcctcagagtcctcatggactacaacaggatgacagaactacttgttatgaagaataagtgacatgatgctcataaagtcctgggtacattctgtaattca

SEQ ID NO: 8 - DNA, 4485 bp, Homo sapiens
gtagacagatccaggctcaccagtcctgtgccactgggcttttggcgttctgcacaaggcctacccgcagatgccatgcctgctcccccagcctaatgggctttgatgggggaagagggtggttcagcctctcacgatgaggaggaaagagcaagtgtcctcctcggacattctccgggttgtgaaatgtgctcgcaggaggcttttcaggcacagaggagccagctggtcgagctgctggtctcagggtccctggaaggcttcgagagtgtcctggactggctgctgtcctgggaggtcctctcctgggaggactacgagggcttccacctcctgggccagcctctctcccacttggccaggcgccttctggacaccgtctggaataagggtacttgggcctgtcagaagctcatcgcggctgcccaagaagcccaggccgacagccagtcccccaagctgcatggctgctgggacccccactcgctccacccagcccgagacctgcagagtcaccggccagccattgtcaggaggctccacagccatgtggagaacatgctggacctggcatgggagcggggtttcgtcagccagtatgaatgtgatgaaatcaggttgccgatcttcacaccgtcccagagggcaagaaggctgcttgatcttgccacggtgaaagcgaatggattggctgccttccttctacaacatgttcaggaattaccagtcccattggccctgcctttggaagctgccacatgcaagaagtatatggccaagctgaggaccacggtgtctgctcagtctcgcttcctcagtacctatgatggagcagagacgctctgcctggaggacatatacacagagaatgtcctggaggtctgggcagatgtgggcatggctggacccccgcagaagagcccagccaccctgggcctggaggagctcttcagcacccctggccacctcaatgacgatgcggacactgtgctggtggtgggtgaggcgggcagtggcaagagcacgctcctgcagcggctgcacttgctgtgggctgcagggcaagacttccaggaatttctctttgtcttcccattcagctgccggcagctgcagtgcatggccaaaccactctctgtgcggactctactctttgagcactgctgttggcctgatgttggtcaagaagacatcttccagttactccttgaccaccctgaccgtgtcctgttaacctttgatggctttgacgagttcaagttcaggttcacggatcgtgaacgccactgctccccgaccgaccccacctctgtccagaccctgctcttcaaccttctgcagggcaacctgctgaagaatgcccgcaaggtggtgaccagccgtccggccgctgtgtcggcgttcctcaggaagtacatccgcaccgagttcaacctcaagggcttctctgaacagggcatcgagctgtacctgaggaagcgccatcatgagcccggggtggcggaccgcctcatccgcctgctccaagagacctcagccctgcacggtttgtgccacctgcctgtcttctcatggatggtgtccaaatgccaccaggaactgttgctgcaggagggggggtccccaaagaccactacagatatgtacctgctgattctgcagcattttctgctgcatgccacccccccagactcagcttcccaaggtctgggacccagtcttcttcggggccgcctccccaccctcctgcacctgggcagactggctctgtggggcctgggcatgtgctgctacgtgttctcagcccagcagctccaggcagcacaggtcagccctgatgacatttctcttggcttcctggtgcgtgccaaaggtgtcgtgccagggagtacggcgcccctggaattccttcacatcactttccagtgcttctttgccgcgttctacctggcactcagtgctgatgtgccaccagctttgctcagacacctcttcaattgtggcaggccaggcaactcaccaatggccaggctcctgcccacgatgtgcatccaggcctcggagggaaaggacagcagcgtggcagctttgctgcagaaggccgagccgcacaaccttcagatcacagcagccttcctggcagggctgttgtcccgggagcactggggcctgctggctgagtgccagacatctgagaaggccctgctccggcgccaggcctgtgcccgctggtgtctggcccgcagcctccgcaagcacttccactccatcccgccagctgcaccgggtgaggccaagagcgtgcatgccatgcccgggttcatctggctcatccggagcctgtacgagatgcaggaggagcggctggctcggaaggctgcacgtggcctgaatgttgggcacctcaagttgacattttgcagtgtgggccccactgagtgtgctgccctggcctttgtgctgcagcacctccggcggcccgtggccctgcagctggactacaactctgtgggtgacattggcgtggagcagctgctgccttgccttggtgtctgcaaggctctgtatttgcgcgataacaatatctcagaccgaggcatctgcaagctcattgaatgtgctcttcactgcgagcaattgcagaagttagctctattcaacaacaaattgactgacggctgtgcacactccatggctaagctccttgcatgcaggcagaacttcttggcattgaggctggggaataactacatcactgccgcgggagcccaagtgctggccgaggggctccgaggcaacacctccttgcagttcctgggattctggggcaacagagtgggtgacgagggggcccaggccctggctgaagccttgggtgatcaccagagcttgaggtggctcagcctggtggggaacaacattggcagtgtgggtgcccaagccttggcactgatgctggcaaagaacgtcatgctagaagaactctgcctggaggagaaccatctccaggatgaaggtgtatgttctctcgcagaaggactgaagaaaaattcaagtttgaaaatcctgaagttgtccaataactgcatcacctacctaggggcagaagccctcctgcaggcccttgaaaggaatgacaccatcctggaagtctggctccgagggaacactttctctctagaggaggttgacaagctcggctgcagggacaccagactcttgctttgaagtctccgggaggatgttcgtctcagtttgtttgtgagcaggctgtgagtttgggccccagaggctgggtgacatgtgttggcagcctcttcaaaatgagccctgtcctgcctaaggctgaacttgttttctgggaacaccataggtcacctttattctggcagaggagggagcatcagtgccctccaggatagacttttcccaagcctacttttgccattgacttcttcccaagattcaatcccaggatgtacaaggacagcccctcctccatagtatgggactggcctctgctgatcctcccaggcttccgtgtgggtcagtggggcccatggatgtgcttgttaactgagtgccttttggtggagaggcccggcctctcacaaaagaccccttaccactgctctgatgaagaggagtacacagaacacataattcaggaagcagctttccccatgtctcgactcatccatccaggccattccccgtctctggttcctcccctcctcctggactcctgcacacgctccttcctctgaggctgaaattcagaatattagtgacctcagctttgatatttcacttacagcacccccaaccctggcacccagggtgggaagggctacaccttagcctgccctcctttccggtgtttaagacatttttggaaggggacacgtgacagccgtttgttccccaagacattctaggtttgcaagaaaaatatgaccacactccagctgggatcacatgtggacttttatttccagtgaaatcagttactcttcagttaagcctttggaaacagctcgactttaaaaagctccaaatgcagctttaaaaaattaatctgggccagaatttcaaacggcctcactaggcttctggttgatgcctgtgaactgaactctgacaacagacttctgaaatagacccacaagaggcagttccatttcatttgtgccagaatgctttaggatgtacagttatggattgaaagtttacaggaaaaaaaattaggccgttccttcaaagcaaatgtcttcctggattattcaaaatgatgtatgttgaagcctttgtaaattgtcagatgctgtgcaaatgttattattttaaacattatgatgtgtgaaaactggttaatatttataggtcactttgttttactgtcttaagtttatactcttatagacaacatggccgtgaactttatgctgtaaataatcagaggggaataaactgttgagtcaaaac

SEQ ID NO: 9 - DNA, 601 bp, Homo sapiens
cttcacatcactttccagtgcttctttgccgcgttctacctggcactcagtgctgatgtgccaccagctttgctcagacacctcttcaattgtggcaggccaggcaactcaccaatggccaggctcctgcccacgatgtgcatccaggcctcggagggaaaggacagcagcgtggcagctttgctgcagaaggccgagccgcacaaccttcagatcacagcagccttcctggcagggctgttgtcccgggagcactggggcctgctggctgagtgccagacatctgagaaggccctgctcyggcgccaggcctgtgcccgctggtgtctggcccgcagcctccgcaagcacttccactccatcccgccagctgcaccgggtgaggccaagagcgtgcatgccatgcccgggttcatctggctcatccggagcctgtacgagatgcaggaggagcggctggctcggaaggctgcacgtggcctgaatgttgggcacctcaagttgacattttgcagtgtgggccccactgagtgtgctgccctggcctttgtgctgcagcacctccggcggcccgtggccctgcagctggactacaactctg

SEQ ID NO: 10 - DNA, 601 bp, Homo sapiens
ctcttgtcagtgagttcctgtccttaagggttagggctgggtagccctctactattctctaagtctgtaatgtaaagccactgaaaactcttgggttaagtttggccatcccacccaaaagatggaggcaggtccactttgctgggaccaggagccccagtgaggccactctgggattgagtggtcctgcccctctggctgggactgcagagggaggaggactgttagttcatgtctagaacacatatcaggtactcactgacactgtctgttgactcttttggccttttcagattctggsgcaacagagtgggtgacgagggggcccaggccctggctgaagccttgggtgatcaccagagcttgaggtggctcaggtaagcttcagagtctatcctgcagttttcttggggagatcaggtgaagagggaggagctggggccagttctgaaggtctttgaactttatttctaccccacaatgttaggcaatggagtaaggaaaaaagaccattggatttcaagagaggacactcgagtctttctgggtgacttggaaatgtcccttgtcctctcagggttttgatacagtatctgtaaat

SEQ ID NO: 11 - DNA, 330 bp, Homo sapiens
  misc_feature (142)..(142): n is a, c, g, or t
gactggctaactcctgcagtctctttaactggacagtttcaagaggaaaaccaagaatccttgaagctcaccattgtatcttcttttccaggttgtccaataactgcatcacctacctaggggcagaagccctcctgcaggncccttgaaaggaatgacaccatcctggaagtctggtaaggcccctgggcaggcctgttttagctctccgaacctcagtttttctatctgtaaaatggggtgacgggagagaggaatggcagaattttgaggatcccttctgattctgacattcagtgagaatgattctgcatgtgaaggatctgattc

SEQ ID NO: 12 - DNA, 21 bp, Homo sapiens
cttccttgcaggactcaccac

SEQ ID NO: 13 - DNA, 22 bp, Homo sapiens
gctgatgtgaaggtgcaaactc

SEQ ID NO: 14 - DNA, 23 bp, Homo sapiens
acctgcttgtcagccagctccgg

SEQ ID NO: 15 - DNA, 24 bp, Homo sapiens
gactacctcatgaagatcctcacc

SEQ ID NO: 16 - DNA, 23 bp, Homo sapiens
tctccttaatgtcacgcacgatt

SEQ ID NO: 17 - DNA, 24 bp, Homo sapiens
cggctacagcttcaccaccacggc